(19) United States
(12) Patent Application Publication
(10) Pub. No.: US 2010/0160623 A1
(43) Pub. Date: Jun. 24, 2010

(54) CYCLODEXTRIN INCLUSION COMPLEXES AND METHODS OF PREPARING SAME

(75) Inventor: Kenneth J. Strassburger, Cincinnati, OH (US)

Correspondence Address: MICHAEL BEST & FRIEDRICH LLP, 100 E WISCONSIN AVENUE, Suite 3300, MILWAUKEE, WI, 53202, US

(73) Assignee: CARGILL, INCORPORATED, Wayzata, MN (US)

(21) Appl. No.: 12/521,341

(22) PCT Filed: Dec. 27, 2007
(86) PCT No.: PCT/US07/88929

§ 371 Date: Feb. 23, 2010

Related U.S. Application Data
(60) Provisional application No. 60/877,489, filed on Dec. 27, 2006.
(60) Provisional application No. 60/877,463, filed on Dec. 27, 2006.

(51) Int. Cl. C07H 3/00

(52) U.S. Cl. 536/123.1

(57) ABSTRACT

The present invention provides a product comprising a guest complexed with a cyclodextrin wherein the guest is more stable in the product and does not degrade as quickly as a product comprising the same guest without a cyclodextrin. In addition, the present invention provides a method of stabilizing guests with a cyclodextrin and reducing the formation of guest degradation products.

### CROSS-REFERENCE TO RELATED APPLICATIONS

This application claims the benefit of U.S. Application Ser. No. 60/877,489, filed on Dec. 27, 2006, and U.S. Application Ser. No. 60/877,463, filed on Dec. 27, 2006, both of which are hereby incorporated by reference.

### BACKGROUND

The following U.S. patents disclose the use of cyclodextrins to complex various guest molecules, and are hereby fully incorporated herein by reference: U.S. Pat. Nos. 4,296,137, 4,296,138 and 4,348,416 to Borden (flavoring material for use in chewing gum, dentifrices, cosmetics, etc.); 4,265,779 to Gandolfo et al. (suds suppressors in detergent compositions); 3,816,393 and 4,054,736 to Hyashi et al. (prostaglandins for use as a pharmaceutical); 3,846,551 to Mifune et al. (insecticidal and acaricidal compositions); 4,024,223 to Noda et al. (menthol, methyl salicylate, and the like); 4,073,931 to Akito et al. (nitro-glycerine); 4,228,160 to Szjetli et al. (indomethacin); 4,247,535 to Bernstein et al. (complement inhibitors); 4,268,501 to Kawamura et al. (anti-asthmatic actives); 4,365,061 to Szjetli et al. (strong inorganic acid complexes); 4,371,673 to Pitha (retinoids); 4,380,626 to Szjetli et al. (hormonal plant growth regulator), 4,438,106 to Wagu et al. (long chain fatty acids useful to reduce cholesterol); 4,474,822 to Sato et al. (tea essence complexes); 4,529,608 to Szjetli et al. (honey aroma), 4,547,365 to Kuno et al. (hair waving active-complexes); 4,596,795 to Pitha (sex hormones); 4,616,008 Hirai et al. (antibacterial complexes); 4,636,343 to Shibanai (insecticide complexes), 4,663,316 to Ninger et al. (antibiotics); 4,675,395 to Fukazawa et al. (hinokitiol); 4,732,759 and 4,728,510 to Shibanai et al. (bath additives); 4,751,095 to Karl et al. (aspartamane); 4,560,571 (coffee extract); 4,632,832 to Okonogi et al. (instant creaming powder); 5,571,782, 5,660,845 and 5,635,238 to Trinh et al. (perfumes, flavors, and pharmaceuticals); 4,548,811 to Kubo et al. (waving lotion); 6,287,603 to Prasad et al. (perfumes, flavors, and pharmaceuticals); 4,906,488 to Pera (olfactants, flavors, medicaments, and pesticides); and 6,638,557 to Qi et al. (fish oils).

Cyclodextrins are further described in the following publications, which are also incorporated herein by reference: (1) Reineccius, T. A., et al. “Encapsulation of flavors using cyclodextrins: comparison of flavor retention in alpha, beta, and gamma types.” Journal of Food Science. 2002; 67(9): 3271-3279; (2) Shiga, H., et al. “Flavor encapsulation and release characteristics of spray-dried powder by the blended encapsulant of cyclodextrin and gum arabic.” Marcel Dekker, Incl., www.dekker.com. 2001; (3) Szente L., et al. “Molecular Encapsulation of Natural and Synthetic Coffee Flavor with β-cyclodextrin.” Journal of Food Science. 1986; 51(4): 1024-1027; (4) Reineccius, G. A., et al. “Encapsulation of Artificial Flavors by β-cyclodextrin.” Perfumer & Flavorist (ISSN 0272-2666) An Allured Publication. 1986: 11(4): 2-6; and (5) Bhandari, B. R., et al. “Encapsulation of lemon oil by paste method using β-cyclodextrin: encapsulation efficiency and profile of oil volatiles.” J. Agric. Food Chem. 1999; 47: 5194-5197.

### SUMMARY

The present invention provides a product comprising a guest complexed with a cyclodextrin and a guest degradation product, the product having a guest to guest degradation product ratio of at least about 5:1 when stored for at least about 30 days at a temperature of at least about 88° F.

The present invention also provides a product comprising a guest complexed with a cyclodextrin, wherein a concentration of a guest the product decreases by no more than about 25% in about 30 days at a temperature of at least about 88° F.

In addition, the present invention provides a product comprising a guest complexed with a cyclodextrin, wherein the guest decreases in concentration over a period of time, and wherein the decrease in concentration of the guest in the product after about 30 days is less than the decrease in concentration of the guest in a control.

Further, the present invention provides a product comprising a guest complexed with a cyclodextrin and a guest degradation product, wherein the guest degradation product is present in a concentration after about 30 days that is less than a concentration of the guest degradation product in a control after about 30 days.

The present invention also provides a product comprising a guest complexed with a cyclodextrin and a guest degradation product, wherein formation of the guest degradation product is reduced by at least about 200% as compared to formation of a guest degradation product in a control.

The present invention provides a product comprising a polyunsaturated fatty acid and a cyclodextrin, wherein the polyunsaturated fatty acid is complexed to the cyclodextrin.

In addition, the present invention provides a method for reducing degradation of a guest in a product over time comprising adding a guest complexed with a cyclodextrin to the product, wherein the guest is complexed with the cyclodextrin in the presence in an emulsifier and wherein the degradation of the guest is reduced by about 25% due to complexation of the guest with the cyclodextrin as compared to a control.

Further, the present invention provides a method for reducing a decrease in concentration of a guest in a product over time comprising adding a guest complexed with a cyclodextrin to the product, wherein the decrease in concentration of the guest is reduced by at last about 25% due to complexation of the guest with a cyclodextrin as compared to a control.

The present invention also provides a method for improving the flavor stability of a product when exposed to light comprising adding a guest complexed with a cyclodextrin to the product, wherein the flavor stability is improved by at least about 25% as compared to a control.

Other features and aspects of the invention will become apparent by consideration of the detailed description and accompanying drawings.

### BRIEF DESCRIPTION OF THE DRAWINGS

FIG. 1 is a schematic illustration of a cyclodextrin molecule having a cavity, and a guest molecule held within the cavity.

FIG. 2 is a schematic illustration of a nano-structure formed by self-assembled cyclodextrin molecules and guest molecules.

FIG. 3 is a schematic illustration of the formation of a diacetyl-cyclodextrin inclusion complex.

FIG. 4 is a schematic illustration of a nano-structure formed by self-assembled cyclodextrin molecules and diacetyl molecules.

FIG. 5 is a schematic illustration of the formation of a citral-cyclodextrin inclusion complex.

FIG. 6 is a schematic illustration of a nano-structure formed by self-assembled cyclodextrin molecules and citral molecules.

FIG. 7 illustrates a degradation mechanism for citral.

FIG. 7A is a schematic illustration of a three-phase model used to represent a guest-cyclodextrin-solvent system.

FIGS. 8-11 illustrate the effect of cyclodextrin on levels of citral and off-notes formed according to Example 20.

FIGS. 12-15 illustrate the effect of cyclodextrin on levels of citral and off-notes formed according to Example 21.

FIGS. 16-17 illustrate the results of a sensory analysis described in Example 34.

FIGS. 18-19 illustrate the effect of cyclodextrin on levels of key note flavors and off-notes formed according to Examples 35-37.

FIG. 20 shows the results of the experiment set forth in Example 38.

FIGS. 21-23 show the bottle beverages of the experiment set forth in Example 40.

FIG. 24-26 show the results for typical offnotes for citral from Example 40A.

FIG. 27 shows the log(P) values for a variety of guests.

FIG. 28 shows stability/method development of citral-cyclodextrin complexes.

FIG. 29 shows stability comparisons of four beverages containing various amounts and forms of citral and cyclodextrin

FIG. 30 shows the stability comparisons of two beverages containing various amounts and forms of citral and cyclodextrin.

FIG. 31 shows the stabilization of citral, color and vitamin content with cyclodextrin

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

In one embodiment, the present invention provides a product comprising at least one guest-cyclodextrin inclusion complex and at least one guest degradation product, the product having a guest to guest degradation product ratio of at least about 10:1 for at least 60 days under accelerated storage conditions, such as 88 degrees F. or 100 degrees F. Suitably, the ratio may be at least about 5:1, about 7:1, about 15:1 or about 20:1. Suitably, the stability may be measured for about 30 days, about 45 days, about 75 days or about 90 days.

In another embodiment, the present invention provides a product comprising a guest complexed with cyclodextrin, the guest having a concentration that decreases in the product over a period of time such as 42 days, the decrease in concentration of the guest in the product being less than a decrease in concentration of the same guest in a second product comprising at least one uncomplexed guest. For example, the decrease in concentration of guest when it is complexed with a cyclodextrin is about 55% less than the decrease in concentration of an uncomplexed guest. Suitably, the decrease in concentration of guest when it is complexed with a cyclodextrin is about 25% less than the decrease in concentration of an uncomplexed guest or about 45% less or about 75% less than the decrease in concentration of an uncomplexed guest. Suitably, the stability may be measured for about 30 days, about 45 days, about 60 days, about 75 days or about 90 days.

In yet another embodiment, the present invention provides a product comprising at least one guest-cyclodextrin inclusion complex, wherein the product contains at least one guest degradation product and wherein concentration of guest degradation product after about 30 days is less than concentration of guest degradation product in a second product comprising at least one uncomplexed guest after about 30 days. Suitably, the stability may be measured for about 45 days, about 60 days, about 75 days or about 90 days.

In a further embodiment, the present invention provides a product comprising a guest-cyclodextrin inclusion complex, wherein a concentration of the guest in the product decreases by no more than about 25% in about 30 days under accelerated storage conditions, such as 88 degrees F. or 100 degrees F. Suitably, the decrease is no more than about 35% or no more than about 50%. Suitably, the stability may be measured for about 45 days, about 60 days, about 75 days or about 90 days.

In an additional embodiment, the present invention provides a product comprising at least one guest-cyclodextrin inclusion complex and at least one guest degradation product and wherein formation of the guest degradation product is reduced by about 500% as compared to formation of a guest degradation product in a second product comprising at least one uncomplexed guest over a period of time. Suitably, the stability may be measured for about 30 days, about 45 days, about 60 days, about 75 days or about 90 days. Suitably, formation of the guest degradation product is reduced by about 200% or by about 250% or about 300% or about 400%.

In yet another embodiment, the present invention also provides a method for reducing the degradation of a guest in a product in response to light exposure, the method comprising: adding a guest complexed with cyclodextrin to the product, the method reducing the degradation of the guest better than the same method using the same product and same guest, except that the guest is not complexed. Degradation can be measured by, e.g., formation of guest degradation products. The formation of guest degradation products can be measured by determining the ratio of guest to guest degradation products at different points in time. Formation can also be measured by calculation of the percentage of guest degradation product present in the product. Formation can also be measured by determination of the area under the curve of the corresponding portion of a gas chromatogram when the samples are analyzed using a gas chromatography-mass spectrometry analysis.

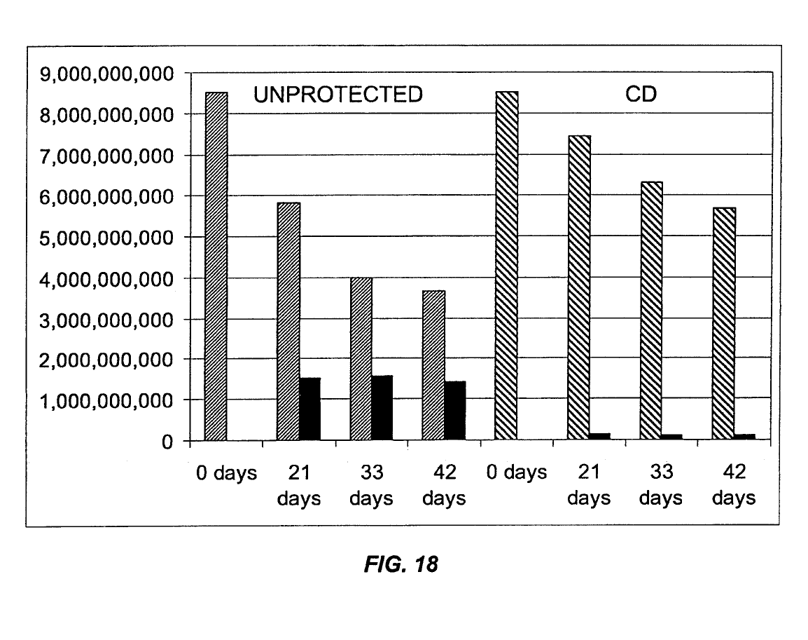

In yet a further embodiment, the present invention provides a method for reducing a decrease in concentration in a guest in a product over time, the method comprising: adding a guest complexed with cyclodextrin to the product, the method reducing the decrease in concentration in the guest in the product over time better than the same method using the same product and same guest, except that the guest is not complexed. The decrease in concentration can be determined by calculating the percentage of guest in the product at different points in time. For example, in FIG. 18, which compares total flavor intensity and offnote development of a protected (right) and un-protected (left) system; the actual values, in raw area counts for flavor intensity are: 5,674,300,000 for protected and 3,662,300,000 for an unprotected system or 155% greater intensity in the protected system compared to the unprotected at 42 days. Also the values for offnote formation are: 108,161,000 in the protected system compared to 1,424,300,000 as seen in the unprotected, which equates to 13.2× the level of offnotes formed in the unprotected system. The system behavior is described algebraically in EQ's 5, 6 and 7 [00132], [00134] and [00137].

In another embodiment, the present invention provides a method for improving the flavor stability of a product when exposed to light, the method comprising adding a guest complexed with cyclodextrin to the product, the method improving the flavor stability of the product when exposed to light over a period of time better than the same method using the same product and same guest, except that the guest is not complexed. The flavor stability can be calculated by, e.g., measuring the formation of guest degradation products over time or measuring the concentration of the guest in the product over time.

In a further embodiment, the invention provides a product comprising a polyunsaturated fatty acid and a cyclodextrin wherein the polyunsaturated fatty acid is complexed with the cyclodextrin.

Each of the methods set forth in paragraphs 42 to 44 may further comprise mixing cyclodextrin and an emulsifier and/or mixing a solvent and a guest to form the guest complexed with cyclodextrin. Alternatively, cyclodextrin, an emulsifier and a thickener may be mixed to form the guest complexed with cyclodextrin. In some embodiments, the cyclodextrin, the emulsifier and thickener may be dry blended. In another embodiment, cyclodextrin, an emulsifier and a thickener may be mixed to form a first mixture, the first mixture is mixed with a solvent to form a second mixture and the second mixture is mixed with the guest to form the guest complexed cyclodextrin. In another embodiment, cyclodextrin, an emulsifier and a thickener may be mixed (e.g., by dry blending) and mixed with a guest (or a solvent and a guest), wherein a weight percent of emulsifier to cyclodextrin is at least about 0.5 wt % and a weight percent of thickener to cyclodextrin is at least about 0.01 wt %. In some embodiments, uncomplexed cyclodextrin is added in molar excess to provide an additional stabilizing effect. The methods are particularly suited for products comprising beverages.

As used herein and in the appended claims, the term “cyclodextrin” can refer to a cyclic dextrin molecule that is formed by enzyme conversion of starch. Specific enzymes, e.g., various forms of cycloglycosyltransferase (CGTase), can break down helical structures that occur in starch to form specific cyclodextrin molecules having three-dimensional polyglucose rings with, e.g., 6, 7, or 8 glucose molecules. For example, α-CGTase can convert starch to α-cyclodextrin having 6 glucose units, β-CGTase can convert starch to β-cyclodextrin having 7 glucose units, and γ-CGTase can convert starch to γ-cyclodextrin having 8 glucose units. Cyclodextrins include, but are not limited to, at least one of α-cyclodextrin, β-cyclodextrin, γ-cyclodextrin, and combinations thereof. β-cyclodextrin is not known to have any toxic effects, is World-Wide GRAS (i.e., Generally Regarded As Safe) and natural, and is FDA approved. α-cyclodextrin and γ-cyclodextrin are also considered natural products and are U.S. and E.U. GRAS.

Suitably, the cyclodextrin may be derivatized. Suitable derivatized cyclodextrins include hydroxyalkylated cyclodextrins, such as 2-hydroxypropyl β-cyclodextrin, 3-hydroxypropyl β-cyclodextrin, 2,3-dihydroxypropyl β-cyclodextrin, and hydroxyethyl β-cyclodextrin, and methylated cyclodextrins, such as methyl β-cyclodextrin.

As used herein and in the appended claims, a “control” is the same product with the same guest, but without a cyclodextrin.

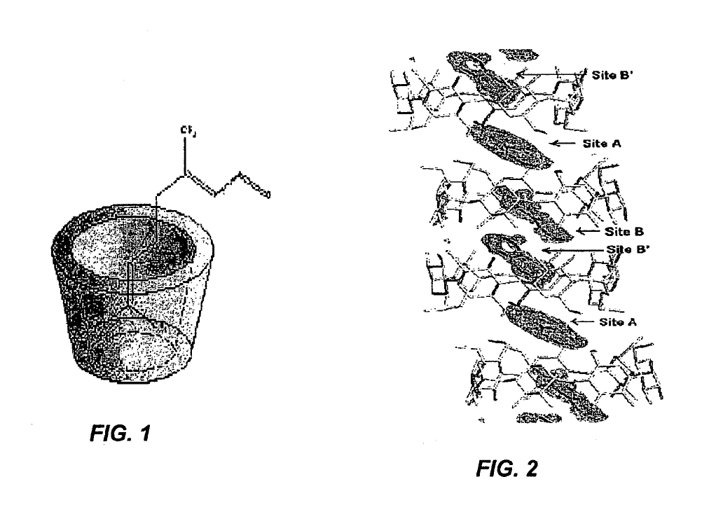

The three-dimensional cyclic structure (i.e., macrocyclic structure) of a cyclodextrin molecule 10 is shown schematically in FIG. 1. The cyclodextrin molecule 10 includes an external portion 12, which includes primary and secondary hydroxyl groups, and which is hydrophilic. The cyclodextrin molecule 10 also includes a three-dimensional cavity 14, which includes carbon atoms, hydrogen atoms and ether linkages, and which is hydrophobic. The hydrophobic cavity 14 of the cyclodextrin molecule can act as a host and hold a variety of molecules, or guests 16, that include a hydrophobic portion to form a cyclodextrin inclusion complex.

As used herein and in the appended claims, the term “guest” can refer to any molecule of which at least a portion can be held or captured within the three dimensional cavity present in the cyclodextrin molecule, including, without limitation, at least one of a flavor, an olfactant, a pharmaceutical agent, a nutraceutical agent (e.g., creatine or vitamins A, C or E) a color agent, and combinations thereof.

Examples of flavors can include, without limitation, flavors based on aldehydes, ketones or alcohols. Examples of aldehyde flavors can include, without limitation, at least one of: acetaldehyde (apple); benzaldehyde (cherry, almond); anisic aldehyde (licorice, anise); cinnamic aldehyde (cinnamon); citral (e.g., geranial, alpha citral (lemon, lime) and neral, beta citral (lemon, lime)); decanal (orange, lemon); ethyl vanillin (vanilla, cream); heliotropine, i.e. piperonal (vanilla, cream); vanillin (vanilla, cream); a-amyl cinnamaldehyde (spicy fruity flavors); butyraldehyde (butter, cheese); valeraldehyde (butter, cheese); citronellal (modifies, many types); decenal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2-6-dimethyl-5-heptenal, i.e. Melonal™ (melon); 2,6-dimethyloctanal (green fruit); 2-dodecenal (citrus, mandarin); and combinations thereof.

Examples of ketone flavors can include, without limitation, at least one of: d-carvone (caraway); l-carvone (spearmint); diacetyl (butter, cheese, “cream”); benzophenone (fruity and spicy flavors, vanilla); methyl ethyl ketone (berry fruits); maltol (berry fruits) menthone (mints), methyl amyl ketone, ethyl butyl ketone, dipropyl ketone, methyl hexyl ketone, ethyl amyl ketone (berry fruits, stone fruits); pyruvic acid (smokey, nutty flavors); acetanisole (hawthorn heliotrope); dihydrocarvone (spearmint); 2,4-dimethylacetophenone (peppermint); 1,3-diphenyl-2-propanone (almond); acetocumene (orris and basil, spicy); isojasmone (jasmine); d-isomethylionone (orris like, violet); isobutyl acetoacetate (brandy-like); zingerone (ginger); pulegone (peppermint-camphor); d-piperitone (minty); 2-nonanone (rose and tea-like); and combinations thereof.

Examples of alcohol flavors can include, without limitation, at least one of anisic alcohol or p-methoxybenzyl alcohol (fruity, peach); benzyl alcohol (fruity); carvacrol or 2-p-cymenol (pungent warm odor); carveol; cinnamyl alcohol (floral odor); citronellol (rose like); decanol; dihydrocarveol (spicy, peppery); tetrahydrogeraniol or 3,7-dimethyl-1-octanol (rose odor); eugenol (clove); p-mentha-1,8dien-7-Oλ or perillyl alcohol (floral-pine); alpha terpineol; mentha-1,5-dien-8-ol 1; mentha-1,5-dien-8-ol 2; p-cymen-8-ol; and combinations thereof.

Examples of olfactants can include, without limitation, at least one of natural fragrances, synthetic fragrances, synthetic essential oils, natural essential oils, and combinations thereof.

Examples of the synthetic fragrances can include, without limitation, at least one of terpenic hydrocarbons, esters, ethers, alcohols, aldehydes, phenols, ketones, acetals, oximes, and combinations thereof.

Examples of terpenic hydrocarbons can include, without limitation, at least one of lime terpene, lemon terpene, limonen dimer, and combinations thereof.

Examples of esters can include, without limitation, at least one of γ-undecalactone, ethyl methyl phenyl glycidate, allyl caproate, amyl salicylate, amyl benzoate, amyl acetate, benzyl acetate, benzyl benzoate, benzyl salicylate, benzyl propionate, butyl acetate, benzyl butyrate, benzyl phenylacetate, cedryl acetate, citronellyl acetate, citronellyl formate, p-cresyl acetate, 2-t-pentyl-cyclohexyl acetate, cyclohexyl acetate, cis-3-hexenyl acetate, cis-3-hexenyl salicylate, dimethylbenzyl acetate, diethyl phthalate, δ-deca-lactone dibutyl phthalate, ethyl butyrate, ethyl acetate, ethyl benzoate, fenchyl acetate, geranyl acetate, γ-dodecalatone, methyl dihydrojasmonate, isobornyl acetate, β-isopropoxyethyl salicylate, linalyl acetate, methyl benzoate, o-t-butylcyclohexyl acetate, methyl salicylate, ethylene brassylate, ethylene dodecanoate, methyl phenyl acetate, phenylethyl isobutyrate, phenylethylphenyl acetate, phenylethyl acetate, methyl phenyl carbinyl acetate, 3,5,5-trimethylhexyl acetate, terpinyl acetate, triethyl citrate, p-t-butylcyclohexyl acetate, vetiver acetate, and combinations thereof.

Examples of ethers can include, without limitation, at least one of p-cresyl methyl ether, diphenyl ether, 1,3,4,6,7,8-hexahydro-4,6,7,8,8-hexamethyl cyclopenta-β-2-benzopyran, phenyl isoamyl ether, and combinations thereof.

Examples of alcohols can include, without limitation, at least one of n-octyl alcohol, n-nonyl alcohol, β-phenylethyldimethyl carbinol, dimethyl benzyl carbinol, carbitol dihydromyrcenol, dimethyl octanol, hexylene glycol linalool, leaf alcohol, nerol, phenoxyethanol, γ-phenyl-propyl alcohol, β-phenylethyl alcohol, methylphenyl carbinol, terpineol, tetraphydroalloocimenol, tetrahydrolinalool, 9-decen-1-ol, and combinations thereof.

Examples of aldehydes can include, without limitation, at least one of n-nonyl aldehyde, undecylene aldehyde, methylnonyl acetaldehyde, anisaldehyde, benzaldehyde, cyclamenaldehyde, 2-hexylhexanal, ahexylcinnamic alehyde, phenyl acetaldehyde, 4-(4-hydroxy-4-methylpentyl)-3-cyclohexene-1-carboxyaldehyde, p-t-butyl-a-methylhydro-cinnamic aldehyde, hydroxycitronellal, α-amylcinnamic aldehyde, 3,5-dimethyl-3-cyclohexene-1-carboxyaldehyde, and combinations thereof.

Examples of phenols can include, without limitation, methyl eugenol.

Examples of ketones can include, without limitation, at least one of 1-carvone, α-damascon, ionone, 4-t-pentylcyclohexanone, 3-amyl-4-acetoxytetrahydropyran, menthone, methylionone, p-t-amycyclohexanone, acetyl cedrene, and combinations thereof.

Examples of the acetals can include, without limitation, phenylacetaldehydedimethyl acetal.

Examples of oximes can include, without limitation, 5-methyl-3-heptanon oxime.

A guest can further include, without limitation, at least one of fatty acids, fatty acid triglycerides, polyunsaturated fatty acids and triglycerides thereof, tocopherols, lactones, terpenes, diacetyl, dimethyl sulfide, proline, furaneol, linalool, acetyl propionyl, cocoa products, natural essences (e.g., orange, tomato, apple, cinnamon, raspberry, etc.), essential oils (e.g., orange, lemon, lime, etc.), sweeteners (e.g., aspartame, neotame, acesulfame-K, saccharin, neohesperidin dihydrochalcone, glycyrrhiza, and stevia derived sweeteners), sabinene, p-cymene, p,a-dimethyl styrene, and combinations thereof.

Examples of polyunsaturated fatty acids (PUFA) can include, without limitation, C18, C20 and C22, omega-3 fatty acids, and C18, C20 and C22, omega-6 fatty acids. For example, suitable polyunsaturated fatty acids include docosahexaenoic acid (DHA), eicosapentaenoic acid (EPA), eicosatetraenoic acid (also known as arachiodonic acid (ARA)), gammalinolenic acid (GLA), stearidonic acid, oleic acid, linoleic acid, and linolenic acids. It is also understood that since PUFAs generally exist in nature as mono, di and tri glycerides, both the free acids and their bound forms are suitable for use in the present invention.

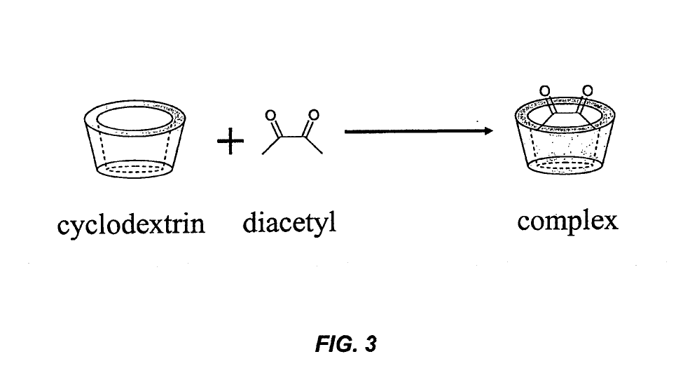

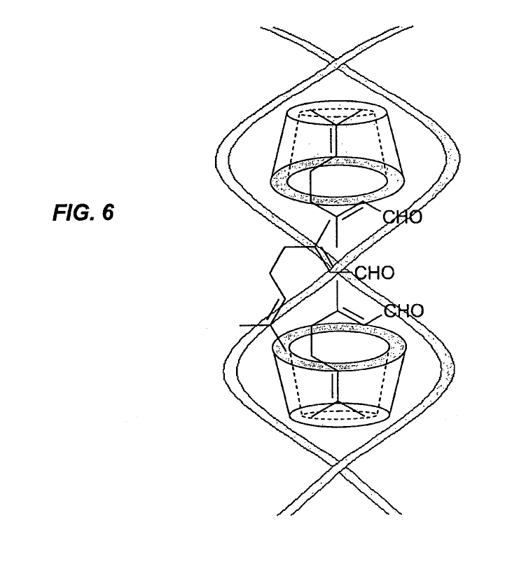

FIG. 3 shows a schematic illustration of the formation of a diacetyl-cyclodextrin inclusion complex, and FIG. 5 shows a schematic illustration of the formation of a citral-cyclodextrin inclusion complex.

As used herein and in the appended claims, the term “guest degradation product” refers to compounds that are formed as the guest decomposes upon exposure to environmental factors, such as light and heat. The presence of the guest degradation product indicates that the concentration of the guest is reduced in a product. For example, if the guest is a flavor, the product loses some of its flavor and may develop an offnote. Offnotes are much more powerful taste agents. Coupled with a loss of flavor intensity, product quality is quickly and dramatically reduced. If the guest is a vitamin or a nutriceutical, the product loses some of the benefits of that vitamin or nutriceutical.

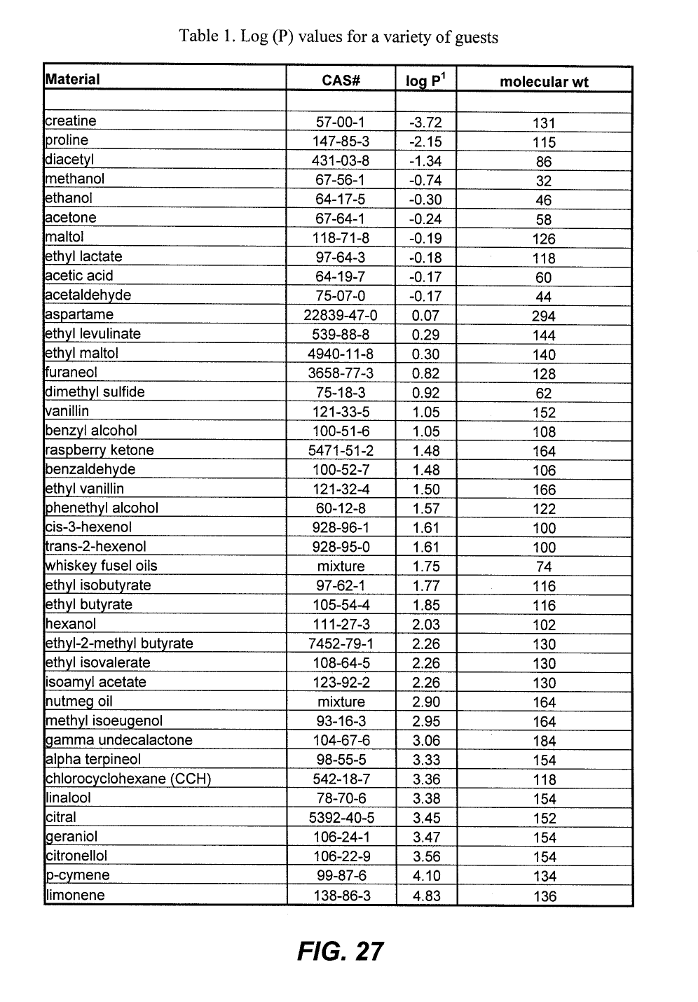

As used herein and in the appended claims, the term “log(P)” or “log(P) value” is a property of a material that can be found in standard reference tables, and which refers to the material's octanol/water partition coefficient. Generally, the log(P) value of a material is a representation of its hydrophilicity/hydrophobicity. P is defined as the ratio of the concentration of the material in octanol to the concentration of the material in water. Accordingly, the log(P) of a material of interest will be negative if the concentration of the material in water is higher than the concentration of the material in octanol. The log(P) value will be positive if the concentration is higher in octanol, and the log(P) value will be zero if the concentration of the material of interest is the same in water as in octanol. Accordingly, guests can be characterized by their log(P) value. For reference, Table 1 shown in FIG. 27 lists log(P) values for a variety of materials, some of which may be guests of the present invention.

Examples of guests having a relatively large positive log(P) value (e.g., greater than about 2) include, but are not limited to, citral, linalool, alpha terpineol, and combinations thereof. Examples of guests having a relatively small positive log(P) value (e.g. less than about 1 but greater than zero) include, but are not limited to, dimethyl sulfide, furaneol, ethyl maltol, aspartame, and combinations thereof. Examples of guests having a relatively large negative log(P) value (e.g., less than about −2) include, but are not limited to, creatine, proline, and combinations thereof. Examples of guests having a relatively small negative log(P) value (e.g., less than 0 but greater than about −2) include, but are not limited to, diacetyl, acetaldehyde, maltol, and combinations thereof.

Log(P) values are significant in many aspects of food and flavor chemistry. A table of log(P) values is provided above. The log(P) values of guests can be important to many aspects of an end product (e.g., foods and flavors). Generally, organic guest molecules having a positive log(P) can be successfully encapsulated in cyclodextrin. In a mixture comprising several guests, competition can exist, and log(P) values can be useful in determining which guests will be more likely to be successfully encapsulated. Maltol and furaneol are examples of two guests that have similar flavor characteristics (i.e., sweet attributes), but which would have different levels of success in cyclodextrin encapsulation because of their differing log(P) values. Log(P) values may be important in food products with a high aqueous content or environment. Compounds with significant and positive log (P) values are, by definition, the least soluble and therefore the first to migrate, separate, and then be exposed to change in the package. The high log(P) value, however, may make them effectively scavenged and protected by addition cyclodextrin in the product. Suitably, the guest has a log(P) of greater than about 1.0 or greater than about 1.50 or greater than about 1.75.

Citral (log(P)=3.45) is a citrus or lemon flavor that can be used in various applications, such as acidic beverages. Acidic beverages can include, but are not limited to lemonade, 7UP® lemon-lime flavored soft drink (registered trademark of Dr Pepper/Seven-Up, Inc.), SPRITES lemon-lime flavored soft drink (registered trademark of The Coca-Cola Company, Atlanta, Ga.), SIERRA MIST® lemon-lime flavored soft drink (registered trademark of Pepsico, Purchase, N.Y.), tea (e.g., LIPTON® and BRISK®, registered trademarks of Lipton), alcoholic beverages, and combinations thereof. Alpha terpineol (log(P)=3.33) is a lime flavor that can be used in similar products as those listed above with respect to citral.

Benzaldehyde (log(P)=1.48) is a cherry flavor that can be used in a variety of applications, including acidic beverages. An example of an acidic beverage that can be flavored with benzaldehyde includes, but is not limited to CHERRY COKE® cherry-cola flavored soft drink (registered trademark of The Coca-Cola Company, Atlanta, Ga.).

Vanillin (log(P)=1.05) is a vanilla flavor that can be used in a variety of applications, including, but not limited to, vanilla-flavored beverages, baked goods, etc., and combinations thereof.

Aspartame (log(P)=0.07) is a non-sucrose sweetener that can be used in variety of diet foods and beverages, including, but not limited to, diet soft drinks. Neotame is also a non-sucrose sweetener that can be used in diet foods and beverages.

Acetaldehyde (log(P)=−0.17) is an apple flavor that can be used in a variety of applications, including, but not limited to, foods, beverages, candies, etc., and combinations thereof.

Creatine (log(P)=−3.72) is a nutraceutical agent that can be used in a variety of applications, including, but not limited to, nutraceutical formulations. Examples of nutraceutical formulations include, but are not limited to, powder formulations that can be combined with milk, water or another liquid, and combinations thereof.

As mentioned above, the cyclodextrin used with the present invention can include α-cyclodextrin, β-cyclodextrin, γ-cyclodextrin, and combinations thereof. In embodiments in which a more hydrophilic guest (i.e., having a smaller log(P) value) is used, α-cyclodextrin may be used (i.e., alone or in combination with another type of cyclodextrin) to improve the encapsulation of the guest in cyclodextrin. For example, a combination of α-cyclodextrin and β-cyclodextrin can be used in embodiments employing relatively hydrophilic guests to improve the formation of a cyclodextrin inclusion complex.

As used herein and in the appended claims, the term “cyclodextrin inclusion complex” refers to a complex that is formed by encapsulating at least a portion of one or more guest molecules with one or more cyclodextrin molecules (encapsulation on a molecular level) by capturing and holding a guest molecule within the three dimensional cavity. The guest can be held in position by van der Waal forces within the cavity by at least one of hydrogen bonding and hydrophilic-hydrophobic interactions. The guest can be released from the cavity when the cyclodextrin inclusion complex is dissolved in water. Cyclodextrin inclusion complexes are also referred to herein as “guest-cyclodextrin complexes.” Because the cavity of cyclodextrin is hydrophobic relative to its exterior, guests having positive log(P) values (particularly, relatively large positive log(P) values) will encapsulate easily in cyclodextrin and form stable cyclodextrin inclusion complexes in an aqueous environment, because the guest will thermodynamically prefer the cyclodextrin cavity to the aqueous environment. In some embodiments, when it is desired to complex more than one guest, each guest can be encapsulated separately to maximize the efficiency of encapsulating the guest of interest.

As used herein and in the appended claims, the term “uncomplexed cyclodextrin” generally refers to cyclodextrin that is substantially free of a guest and has not formed a cyclodextrin inclusion complex. Cyclodextrin that is “substantially free of a guest” generally refers to a source of cyclodextrin that includes a large fraction of cyclodextrin that does not include a guest in its cavity.

As used herein and in the appended claims, the term “hydrocolloid” generally refers to a substance that forms a gel with water. A hydrocolloid can include, without limitation, at least one of xanthan gum, pectin, gum arabic (or gum acacia), tragacanth, guar, carrageenan, locust bean, and combinations thereof.

As used herein and in the appended claims, the term “pectin” refers to a hydrocolloidal polysaccharide that can occur in plant tissues (e.g., in ripe fruits and vegetables). Pectin can include, without limitation, at least one of beet pectin, fruit pectin (e.g., from citrus peels), and combinations thereof. The pectin employed can be of varying molecular weight.

Cyclodextrin inclusion complexes of the present invention can be used in a variety of applications or end products, including, without limitation, at least one of foods (e.g., beverages, such as carbonated beverages, citrus drinks, lemonade, juices, soft drinks, sports drinks, vitamin fortified drinks etc., salad dressings, popcorn, cereal, apple sauce, coffee, cookies, brownies, gelatins, other desserts, other baked goods, seasonings, etc.), chewing gums, dentifrices, such as toothpastes and mouth rinses, candy, flavorings, fragrances, pharmaceuticals (e.g. cough syrup preparations, etc.), nutraceuticals, cosmetics, agricultural applications (e.g., herbicides, pesticides, etc.), photographic emulsions, and combinations thereof. In some embodiments, cyclodextrin inclusion complexes can be used as intermediate isolation matrices to be further processed, isolated and dried (e.g., as used with waste streams).

Cyclodextrin inclusion complexes can be used to enhance the stability of the guest, convert it to a free flowing powder, or otherwise modify its solubility, delivery or performance. The amount of the guest molecule that can be encapsulated is directly related to the molecular weight of the guest molecule. In some embodiments, one mole of cyclodextrin encapsulates one mole of guest. According to this mole ratio, and by way of example only, in embodiments employing diacetyl (molecular weight of 86 Daltons) as the guest, and β-cyclodextrin (molecular weight 1135 Daltons), the maximum theoretical retention is (86/(86+1135))×100=7.04 wt %.

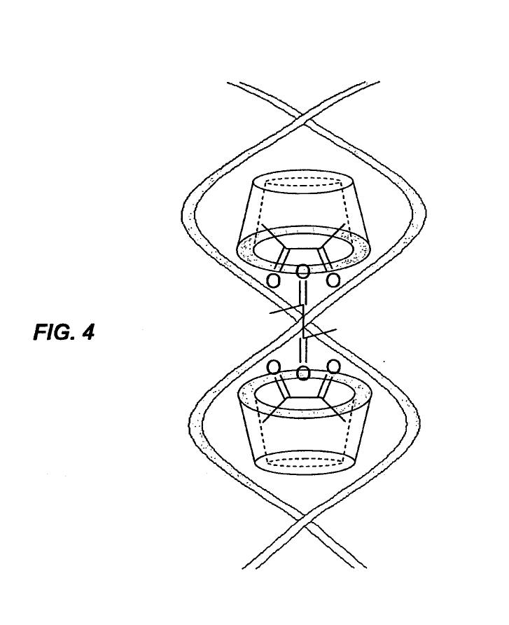

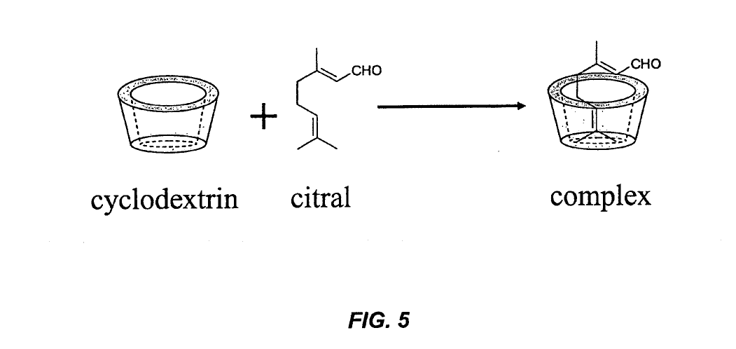

In some embodiments, cyclodextrin can self-assemble in solution to form a nano-structure, such as the nano-structure 20 illustrated in FIG. 2, that can incorporate three moles of a guest molecule to two moles of cyclodextrin molecules. For example, in embodiments employing diacetyl as the guest, a 10.21 wt % retention of diacetyl is possible, and in embodiments employing citral as the guest, a wt % retention of citral of at least 10 wt % is possible (e.g., 10-14 wt % retention). FIG. 4 shows a schematic illustration of a nano-structure than can form between three moles of diacetyl molecules and two moles of cyclodextrin molecules. FIG. 6 shows a schematic illustration of a nano-structure than can form between three moles of citral molecules and two moles of cyclodextrin molecules. Other complex enhancing agents, such as pectin, can aid in the self-assembly process, and can maintain the 3:2 mole ratio of guest:cyclodextrin throughout drying. In some embodiments, because of the self-assembly of cyclodextrin molecules into nano-structures, a 5:3 mole ratio of guest:cyclodextrin is possible.

Cyclodextrin inclusion complexes form in solution. The drying process temporarily locks at least a portion of the guest in the cavity of the cyclodextrin and can produce a dry, free flowing powder comprising the cyclodextrin inclusion complex.

The hydrophobic (water insoluble) nature of the cyclodextrin cavity will preferentially trap like (hydrophobic) guests most easily at the expense of more water-soluble (hydrophilic) guests. This phenomenon can result in an imbalance of components as compared to typical spray drying and a poor overall yield.

In some embodiments of the present invention, the competition between hydrophilic and hydrophobic effects is avoided by selecting key ingredients to encapsulate separately. For example, in the case of butter flavors, fatty acids and lactones form cyclodextrin inclusion complexes more easily than diacetyl. However, these compounds are not the key character impact compounds associated with butter, and they will reduce the overall yield of diacetyl and other water soluble and volatile ingredients. In some embodiments, the key ingredient in butter flavor (i.e., diacetyl) is maximized to produce a high impact, more stable, and more economical product. By way of further example, in the case of lemon flavors, most lemon flavor components will encapsulate equally well in cyclodextrin. However, terpenes (a component of lemon flavor) have little flavor value, and yet make up approximately 90% of a lemon flavor mixture, whereas citral is a key flavor ingredient for lemon flavor. In some embodiments, citral is encapsulated alone. By selecting key ingredients (e.g., diacetyl, citral, etc.) to encapsulate separately, the complexity of the starting material is reduced, allowing optimization of engineering steps and process economics.

In some embodiments, the inclusion process for forming the cyclodextrin inclusion complex is driven to completion by adding a molar excess of the guest. For example, in some embodiments (e.g., when the guest used is diacetyl), the guest can be combined with the cyclodextrin in a 3:1 molar ratio of guest: cyclodextrin. In some embodiments, using a molar excess of guest in forming the complex not only drives the formation of the cyclodextrin inclusion complex, but can also make up for any loss of guest in the process, e.g., in embodiments employing a volatile guest.

In some embodiments, the viscosity of the suspension, emulsion or mixture formed by mixing the cyclodextrin and guest molecules in a solvent is controlled, and compatibility with common spray drying technology is maintained without other adjustments, such as increasing the solids content. An emulsifier (e.g., a thickener, gelling agent, polysaccharide, hydrocolloid, gums such as xanthan, and polyglucoronic acids and their derivatives) can be added to maintain intimate contact between the cyclodextrin and the guest, and to aid in the inclusion process. Particularly, low molecular weight hydrocolloids can be used. One preferred hydrocolloid is pectin, especially beet pectin. Emulsifiers can aid in the inclusion process without requiring the use of high heat or co-solvents (e.g., ethanol, acetone, isopropanol, etc.) to increase solubility.

In some embodiments, the water content of the suspension, emulsion or mixture is reduced to essentially force the guest to behave as a hydrophobic compound. This process can increase the retention of even relatively hydrophilic guests, such as acetaldehyde, diacetyl, dimethyl sulfide, etc. Reducing the water content can also maximize the throughput through the spray dryer and reduce the opportunity of volatile guests blowing off in the process, which can reduce overall yield.

In some embodiments of the present invention, a cyclodextrin inclusion complex can be formed by the following process, which may include some or all of the following steps:

(1) Dry blending cyclodextrin and an emulsifier (e.g., pectin);

(2) Combining the dry blend of cyclodextrin and the emulsifier with a solvent such as water in a reactor, and agitating;

(3) Adding the guest and stirring (e.g., for approximately 5 to 8 hours);

(4) Cooling the reactor (e.g., turning on a cooling jacket);

(5) Stirring the mixture (e.g., for approximately 12 to 36 hours);

(6) Emulsifying (e.g., with an in-tank lightning mixer or high shear drop-in mixer); and

(7) Drying the cyclodextrin inclusion complex to form a powder.

These steps need not necessarily be performed in the order listed. In addition, the above process has proved to be very robust in that the process can be performed using variations in temperature, time of mixing, and other process parameters.

In some embodiments, step 1 in the process described above can be accomplished using an in-tank mixer in the reactor to which the hot water will be added in step 2. For example, in some embodiments, the process above is accomplished using a 1000 gallon reactor equipped with a jacket for temperature control and an inline high shear mixer, and the reactor is directly connected to a spray drier. In some embodiments, the cyclodextrin and emulsifier can be dry blended in a separate apparatus (e.g., a ribbon blender, etc.) and then added to the reactor in which the remainder of the above process is completed.

A variety of weight percentages of an emulsifier to cyclodextrin can be used, including, without limitation, an emulsifier:cyclodextrin weight percentage of at least about 0.5%, particularly, at least about 1%, and more particularly, at least about 2%. In addition, an emulsifier:cyclodextrin weight percentage of less than about 10% can be used, particularly, less than about 6%, and more particularly, less than about 4%.

Step 2 in the process described above can be accomplished in a reactor that is jacketed for heating, cooling, or both. In some embodiments, the combining and agitating can be performed at room temperature. In some embodiments, the combining and agitating can be performed at a temperature greater than room temperature. The reactor size can be dependent on the production size. For example, a 100 gallon reactor can be used. The reactor can include a paddle agitator and a condenser unit. In some embodiments, step 1 is completed in the reactor, and in step 2, hot deionized water is added to the dry blend of cyclodextrin and pectin in the same reactor.

Step 3 can be accomplished in a sealed reactor, or the reactor can be temporarily exposed to the environment while the guest is added, and the reactor can be re-sealed after the addition of the guest. Heat can be added when the guest is added and during the stirring of step 3. For example, in some embodiments, the mixture is heated to about 55-60 degrees C.

Step 4 can be accomplished using a coolant system that includes a cooling jacket. For example, the reactor can be cooled with a propylene glycol coolant and a cooling jacket.

The agitating in step 2, the stirring in step 3, and the stirring in step 5 can be accomplished by at least one of shaking, stirring, tumbling, and combinations thereof.

In step 6, the mixture of the cyclodextrin, emulsifier, water and guest can be emulsified using at least one of a high shear mixer (e.g., a ROSS-brand mixer (e.g., at 10,000 RPM for 90 seconds), or a SILVERSTON-brand mixer (e.g., at 10,000 RPM for 5 minutes)), a lightning mixer, or simple mixing followed by transfer to a homogenization pump that is part of a spray dryer, and combinations thereof.

Step 7 in the process described above can be accomplished by at least one of air drying, vacuum drying, spray drying (e.g., with a nozzle spray drier, a spinning disc spray drier, etc.), oven drying, and combinations thereof.

In some embodiments, the complexation process can utilize a paste method and the mixture can be dried as described above. In other embodiments, the mixture can be appropriately diluted for spray drying. Different methods for improving the manufacturability and stability of a cyclodextrin inclusion complex, including the formation of a liquid or emulsion form comprising the cyclodextrin inclusion complex, are the subject matter of co-pending applications, PCT/US2006/012529 and PCT/US2006/012528, the entire contents of which are incorporated herein by reference.

As mentioned above, the encapsulation of the guest molecule can provide isolation of the guest molecule from interaction and reaction with other components that would cause off note formation; and stabilization of the guest molecule against degradation (e.g., hydrolysis, oxidation, etc.). Stabilization of the guest against degradation can improve or enhance the desired effect or function (e.g., taste, odor, etc.) of a resulting commercial product that includes the encapsulated guest.

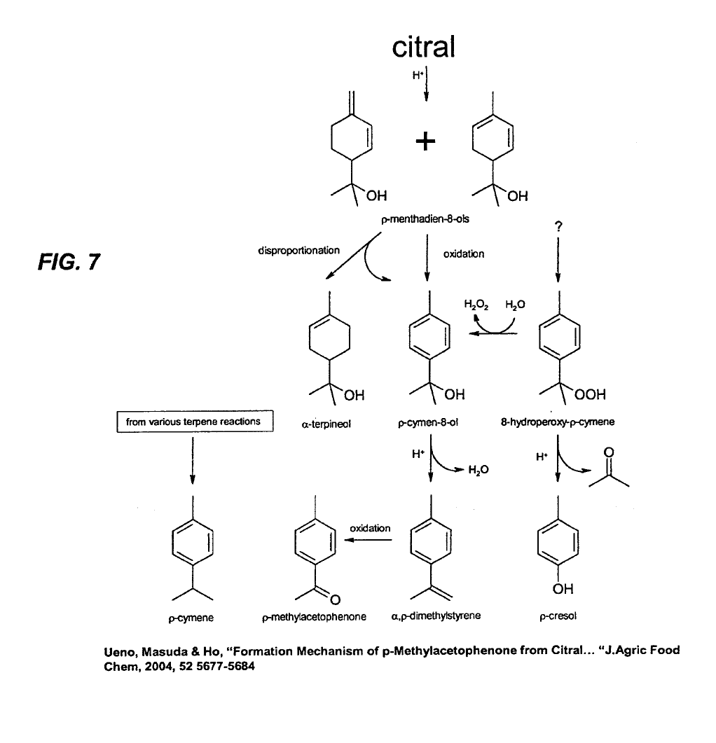

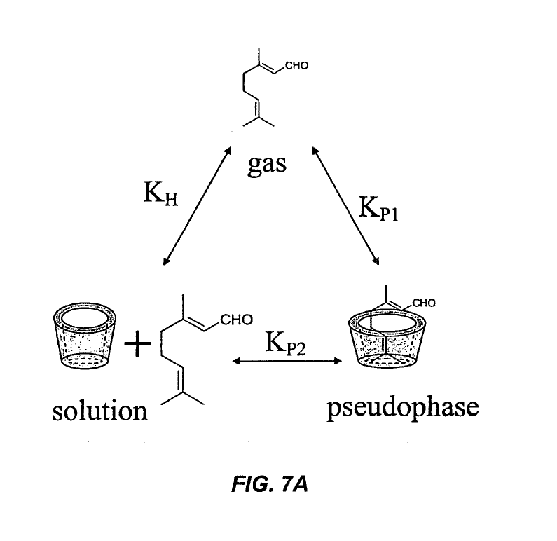

Many guests can degrade and create off-notes that can detract from a main or desired effect or function. For example, many flavors or olfactants can degrade and create off-note flavors or odors that can detract from the desired flavor or odor of a commercial product. Guests can also be degraded by means of photo-oxidation. By way of example, FIG. 7 shows the degradation mechanism of citral. The rate of degradation of the guest (i.e., the rate of formation of off-note(s)) is generally governed by the following generic kinetic rate equation:

$$Rate \approx \frac{{[ offnote ]}^{z}}{{[ guest ]}^{x} \cdot {[ RC ]}^{y }}$$

where [guest] refers to the molar concentration of guest in a solution, [RC] refers to the molar concentration of a reactive compound in a solution responsible for reacting with and degrading the guest (e.g., an acid), and [offnote] refers to the molar concentration of off-notes formed. The powers x, y and z represent kinetic order, depending on the reaction that occurs between a guest of interest and the corresponding reactive compound(s) present in solution to produce off-notes. Thus, the rate of degradation of the guest is proportional to the product of the molar concentrations of the guest and any reactive compounds, raised to a power determined by the kinetic order of the reaction.

For example, the following equation represents the degradation of citral in an acidic solution to form off-notes at any given temperature and concentration:

$$\frac{{[ offnote ]}^{z}}{{[ citral ]}^{x} \times {[ {H}^{+} ]}^{y}} = κ$$

where, based on the degradation mechanism of citral shown in FIG. 7,

$$[ offnote ] = \sum {κ [ p - menthadien - 8 - ol ]}^{{RP}_{1}} + {κ [ p - cymen - 8 - ol ]}^{{RP}_{2}} + … + {κ [ p - methylacetophenone ]}^{{RP}_{n}}$$

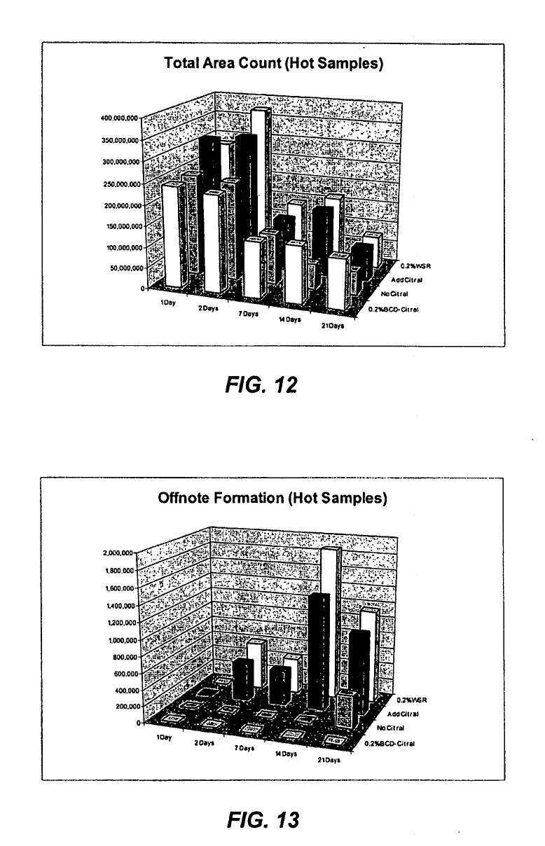

Any of the above-mentioned guests can be protected and stabilized in this manner. For example, cyclodextrin can be used to protect and/or stabilize a variety of guest molecules to enhance the desired effect or function of a product, including, but not limited to, the following guest molecules: citral, benzaldehyde, alpha terpineol, vanillin, aspartame, neotame, acetaldehyde, creatine, and combinations thereof. An example of this phenomenon is described in Example 21 and shown in Table 2 and FIGS. 12-15. Specifically, this phenomenon was demonstrated by comparing samples 1BH3, 1BH4, and 1BH5, all with added citral; and samples 3FH3, 3FH4 and 3FH5, all with water-soluble rosemary (WSR) with the BCD samples. Mentha 1,5-dien-8-ol was converted to p-cymene-8-ol in the 1BH and 3FH samples, and it was observed that the of concentration of mentha 1,5-dien-8-ol, for example, decreased, and the concentration of p-cymene-8-ol increased. However, these reactions or changes did not occur in the protected BCD samples.

A “guest stabilizing system” can refer to any system which stabilizes a guest (or guests) of interest and protects the guest from degradation. The present invention includes several embodiments of guest stabilizing systems, as will be described in greater detail below.

The protection and/or stabilization of a guest can be accomplished by providing an excess of cyclodextrin (e.g., uncomplexed cyclodextrin) to the final powder product of the cyclodextrin inclusion complex. In other words, dry blending uncomplexed cyclodextrin with the dry powder that is formed in step 7 of the process described above can produce a dry, free-flowing powder (referred to herein as “guest-cyclodextrin/cyclodextrin blend”) with a desired amount of guest and cyclodextrin (i.e., including excess uncomplexed cyclodextrin) that can be used in a variety of applications or commercial products. The proportion of a guest-cyclodextrin complex in a guest-cyclodextrin/cyclodextrin blend depends on the potency (e.g., flavor value if the guest is a flavor) of the guest, and the desired effect in the final product. The excess uncomplexed cyclodextrin in the guest-cyclodextrin/cyclodextrin blend acts to protect and/or stabilize the guest (including from photo-oxidation) when the guest-cyclodextrin/cyclodextrin blend is added to, or used in, a product of interest. For example, a flavor powder including a guest-cyclodextrin/cyclodextrin blend can be effective in decreasing the rate of degradation of the flavor in beverage applications while providing an appropriate flavor profile to that beverage.

A variety of systems can be employed to add excess uncomplexed cyclodextrin for protection and/or stabilization of the guest. In some embodiments, the guest-cyclodextrin/cyclodextrin blend is added as a dry powder to a final product (e.g., in a weight percentage of ranging from about 0.05 wt % to about 0.50 wt % of guest-cyclodextrin/cyclodextrin blend to product, particularly, from about 0.15 wt % to about 0.30 wt %, and more particularly, about 0.2 wt %).

In some embodiments, if solubility of the powder permits, the guest-cyclodextrin/cyclodextrin blend is added to a liquid product, emulsion or emulsion-compatible product (e.g., a flavor emulsion), which is then added to the final product (e.g., in a weight percentage of ranging from about 0.05 wt % to about 0.50 wt % of guest-cyclodextrin/cyclodextrin blend to product, particularly, from about 0.15 wt % to about 0.30 wt %, and more particularly, about 0.2 wt %, such that the weight percentage of the guest achieves a desired flavor level in the final product. In some embodiments, the excess uncomplexed cyclodextrin can be added to the composition comprising the cyclodextrin inclusion complex that is formed in step 6, thereby skipping step 7 (the drying step) and forming a stable emulsion or emulsion-compatible product that can be added to the final product in the range of weight percentages listed above. The emulsion-compatible product can be added to another final product (e.g., a beverage, a salad dressing, a dessert, and/or a seasoning, etc.). In some embodiments, the emulsion-compatible product can be provided in the form of, or be added to, a syrup or a coating mix, which can be sprayed onto a substrate as a stable coating (e.g., a flavor emulsion sprayed onto cereal, a dessert, a seasoning, nutritional bars, and/or snack foods such as pretzels, chips, etc.).

Providing the cyclodextrin inclusion complex in a liquid form can, but need not, have several advantages. First, the liquid form can be more familiar and user friendly for beverage customers who are accustomed to adding flavor compositions to their beverages in the form of a liquid concentrate. Second, the liquid form can be easily sprayed onto dry food products including those listed above to achieve an evenly-distributed and stable coating that includes the flavor composition. Unlike existing spray-on applications, the sprayed-on flavor composition comprising the cyclodextrin inclusion complex would not require the typical volatile solvents or additional coatings or protective layers to maintain the flavor composition on that dry substrate. Third, cyclodextrin can extend the shelf-life of such food products, because cyclodextrin is not hygroscopic, and thus will not lead to staleness, flatness, or reduced freshness of the base food product or beverage. Fourth, drying processes can be costly, and some guest (e.g., free guest or guest present in a cyclodextrin inclusion complex) can be lost during drying, which can make the drying step difficult to optimize and perform economically. For these reasons and others that are not specifically mentioned here, providing the cyclodextrin inclusion complex in a liquid form in some embodiments can be beneficial. The emulsion form of the cyclodextrin inclusion complex can be added to a final product (e.g., a beverage or food product) to impart the appropriate guest profile (e.g., flavor profile) to the final product, while ensuring that the cyclodextrin in the final product is within the legal limits for that given product (e.g., no greater than 0.2 wt % of some products, or no greater than 2 wt % of some products).

Because there is an equilibrium that is established between encapsulation of the guest with the cyclodextrin and free (or uncomplexed) guest molecules and cyclodextrin molecules, adding excess uncomplexed cyclodextrin to a system can force the equilibrium to encapsulation of the guest. As described above, decreasing the amount of free guest in a system decreases the rate of degradation of the guest and the rate of formation of off-notes. In addition, especially in beverage or other liquid applications, the guest may prefer, thermodynamically and/or kinetically, to be encapsulated in cyclodextrin over being unencapsulated. This phenomenon can be exaggerated by adding excess uncomplexed cyclodextrin. It is also possible that the small amount of off-note molecules that are formed, if any, may become encapsulated in cyclodextrin, and become essentially “masked” from the final product. In other words, in some embodiments, because of the chemical makeup of the off-notes, the off-notes may bind very stably with cyclodextrin, which can lead to a masking effect of any off-notes that may be formed. Thus, in some embodiments, the excess uncomplexed cyclodextrin may act as a scavenger to mask or isolate other water-miscible components in a system that may interfere with desired effects or functions of a product.

FIG. 7A illustrates a three-phase model that represents a guest-cyclodextrin-solvent system. The guest used in FIG. 7A is citral, and the solvent used is water, but it should be understood that citral and water are shown in FIG. 7A for the purpose of illustration only. One of ordinary skill in the art, however, will understand that the three-phase model shown in FIG. 7A can be used to represent a wide variety of guests and solvents. Additional information regarding a three-phase model similar to the one illustrated in FIG. 7 can be found in Lantz et al., “Use of the three-phase model and headspace analysis for the facile determination of all partition/association constants for highly volatile solute-cyclodextrin-water systems,” Anal Bioanal Chem (2005) 383: 160-166, which is incorporated herein by reference.

This three-phase model can be used to explain the phenomena that occur (1) during formation of the cyclodextrin inclusion complex, (2) in a beverage application of the cyclodextrin inclusion complex, and/or (3) in a flavor emulsion. The flavor emulsion can include, for example, the slurry formed in step 5 or 6 in the process described above prior to or without drying, or a slurry formed by resuspending a dry powder comprising a cyclodextrin inclusion complex in a solvent. Such a flavor emulsion can be added to a beverage application (e.g., as a concentrate), or sprayed onto a substrate, as described above.

As shown in FIG. 7A, there are three phases in which the guest can be present, namely, the gaseous phase, the aqueous phase, and the cyclodextrin phase (also sometimes referred to as a “pseudophase”). Three equilibria, and their associated equilibrium constants (i.e., K_{H}, K_{P1} and K_{P2}) are used to describe the presence of the guest in these three phases:

$$\begin{matrix}{S}_{( g )} {}^{{K}_{H}} {S}_{( aq )} ; \\ {K}_{H} = \frac{{C}_{S}^{aq}}{{P}_{S}} ( based on {Henry}^{′} s Law \text{:} {K}_{H} = \frac{{C}_{S}}{{P}_{S}} ) & ( 1 ) \\ {S}_{( g )} {}^{{K}_{P 1}} {S}_{( CD )} ; {K}_{P 1} = \frac{{C}_{S}^{CD}}{{P}_{S}} & ( 2 ) \\ {S}_{( aq )} {}^{{K}_{P 2}} {S}_{( CD )} ; {K}_{P 2} = \frac{{C}_{S}^{CD}}{{C}_{S }^{aq}} & ( 3 ) \\ {K}_{H} = \frac{{K}_{P 1}}{{K}_{P 2}} & ( 4 )\end{matrix}$$

wherein “S” represents the solute (i.e., the guest) of the system in the corresponding phase of the system which is denoted in the subscript, “g” represents the gaseous phase, “aq” represents the aqueous phase, “CD” represents the cyclodextrin phase, “C_{S}” represents the concentration of the solute in the corresponding phase (i.e., aq or CD, denoted in the superscript), and “P_{S}” represents the partial pressure of the solute in the gaseous phase.

To account for all of the guest in the three-phase system shown in FIG. 7A, it follows that the total number of moles of guest (nₛ^{total}) can be represented by the following equation:

n_{S}^{total}=n_{S}^{g}+n_{S}^{aq}+n_{S}^{CD}. (5)

To account for any loss of the guest in a product (e.g., a beverage or flavor emulsion) at steady state, the total number of moles of guest available for sensation (nₛ^{taste}; e.g., for taste in a beverage or flavor emulsion) can be represented by the following equation:

n_{S}^{taste}=n_{S}^{g}+n_{S}^{aq}+n_{S}^{CD}−ƒ_{(P)} (6)

wherein ƒ_{(P)} is a partitioning function that represents any migration (or loss) of the guest, for example, through a barrier or container (e.g., a plastic bottle formed of polyethylene or polyethylene terephthalate (PET)) in which the beverage of flavor emulsion is contained.

For guests having a large positive log(P) value, encapsulation of the guest in cyclodextrin will be thermodynamically favored (i.e., K_{P1} and K_{P2} will be greater than 1), and the following relationship will occur:

n_{S}^{CD}>>n_{S}^{aq}>n_{S}^{g}>ƒ_{(P)} (7)

such that the majority of the guest present in the system will be in the form of a cyclodextrin inclusion complex. Not only will the amount of free guest in the aqueous and gaseous phases be minimal, but also the migration of guest through the barrier or container will be minimized. Accordingly, the majority of the guest available for sensation will be present in the cyclodextrin phase, and the total number of moles of guest available for sensation (nₛ^{taste}) can be approximated as follows:

n_{S}^{taste}≈n_{S}^{CD} (8)

The formation of the cyclodextrin inclusion complex in solution between the guest and the cyclodextrin can be more completely represented by the following equation:

$${S}_{( aq )} + {CD}_{( aq )} {}^{{K}_{P 2}} S \cdot {CD}_{( aq )} ; {K}_{P 2} = \frac{{[ S \cdot CD ]}_{( aq )}}{{{[ S ]}_{( aq )} [ CD ]}_{( aq )}} \qquad ( 9 )$$

Empirically, the data supporting the present invention has shown that the log(P) value of the guest can be a factor in the formation and stability of the cyclodextrin inclusion complex. That is, empirical data has shown that the equilibrium shown in equation 9 above is driven to the right by the net energy loss accompanied by the encapsulation process in solution, and that the equilibrium can be at least partially predicted by the log(P) value of the guest of interest. It has been found that log(P) values of the guests can be a factor in end products with a high aqueous content or environment. For example, guests with relatively large positive log(P) values are typically the least water-soluble and can migrate and separate from an end product, and can be susceptible to a change in the environment within a package. However, the relatively large log(P) value can make such guests effectively scavenged and protected by the addition of cyclodextrin to the end product. In other words, in some embodiments, the guests that have traditionally been the most difficult to stabilize can be easy to stabilize using the methods of the present invention.

To account for the effect of the log(P) value of the guest, the equilibrium constant (K_{P2}′) that represents the stability of the guest in a system can be represented by the following equation:

$${K}_{P 2}^{′} = log ( P ) \frac{{[ S \cdot CD ]}_{( aq )}}{{{[ S ]}_{( aq )} [ CD ]}_{( aq )}} \qquad ( 10 )$$

wherein log(P) is the log(P) value for the guest (S) of interest in the system. Equation 10 establishes a model that takes into account a guest's log(P) value. Equation 10 shows how a thermodynamically stable system can result from first forming a cyclodextrin inclusion complex with a guest having a relatively large positive log(P) value. For example, in some embodiments, a stable system (i.e., a guest stabilizing system) can be formed using a guest having a positive log(P) value. In some embodiments, a stable system can be formed using a guest having a log(P) value of at least about +1. In some embodiments, a stable system can be formed using a guest having a log(P) value of at least about +2. In some embodiments, a stable system can be formed using a guest having a log(P) value of at least about +3. Furthermore, one can see how a thermodynamically stable system can result not only by using a guest having a positive log(P) value, but also by adding additional, uncomplexed cyclodextrin to that cyclodextrin inclusion complex to further favor the right side of the equilibrium shown in equation 9 above, and to increase the ratio of complexed guest to free, or uncomplexed, guest to further stabilize the guest from degradation.

While log(P) values can be good empirical indicators and are available from several references, another important criteria is the binding constant for a particular guest (i.e., once a complex forms, how strongly is the guest bound in the cyclodextrin cavity). Unfortunately, the binding constant for a guest is determined experimentally. In the case of limonene and citral, for example, citral can form a much stronger complex, even though the log(P) values are similar. As a result, even in the presence of high limonene concentrations, citral is preferentially protected until consumption, because of its higher binding constant. This is an unexpected benefit and is not directly predicted from the current scientific literature.

In some embodiments of the present invention, as supported by equation 10, the guest is added to a product, system or application (e.g., a beverage) in an uncomplexed form, and uncomplexed cyclodextrin is added to that same product, system or application. As suggested by equation 10, the stability of the guest in such a system (and the guest's protection from degradation) will be at least partially dependent on the log(P) value of the guest. For example, a guest can be added to a system to obtain a desired concentration of guest in the system, and uncomplexed cyclodextrin can be added to the system to stabilize the guest and protect the guest from degradation. In some embodiments, the concentration of the guest in the system is at least about 1 ppm, particularly, at least about 5 ppm, and more particularly, at least about 10 ppm. In some embodiments, the concentration of the guest in the system is less than about 200 ppm, particularly, less than about 150 ppm, and more particularly, less than about 100 ppm. In some embodiments, the overall concentration of citrus components, for example, can exceed 1000 ppm (e.g., when limonene is present). However, this has not proved an impediment to the stabilization/protection scheme of the present invention.

In some embodiments, the cyclodextrin is added to the system in a molar ratio of cyclodextrin:guest of greater than 1:1. As shown in equation 10, stabilization of the guest in the system by cyclodextrin can be predicted by the log(P) value of the guest. In some embodiments, the guest chosen has a positive log(P) value. In some embodiments, the guest has a log(P) value of greater than about +1. In some embodiments, the guest has a log(P) value of greater than about +2. In some embodiments, the guest has a log(P) value of greater than about +3.

Whether the product, system or application includes a free/uncomplexed guest, or a cyclodextrin-encapsulated guest, the guest can be added to achieve a desired concentration of the guest in the final product, system or application, and the uncomplexed cyclodextrin can be added to the product, system or application to maintain the total weight percentage of cyclodextrin within legal limits. For example, in some embodiments, the weight percentage of cyclodextrin to the system ranges from about 0.05 wt % to about 0.50 wt %, particularly, from about 0.15 wt % to about 0.30 wt %, and more particularly, about 0.2 wt %. In some embodiments, the uncomplexed cyclodextrin is combined with the guest and then added to the system. In some embodiments, the uncomplexed cyclodextrin is added directly to the system separately from the guest. Example 20 illustrates the stabilizing effects of uncomplexed α-cyclodextrin or β-cyclodextrin added to a solution comprising citral. As explained in Example 20, the citral is protected from degradation and off-note formation is inhibited. Equation 10 suggests that the stabilizing effect of citral can be at least partially due to the relatively large log(P) value of citral (i.e., 3.45).

By taking into account the log(P) of the guest, it is possible to predict the stability of the guest in a system that comprises cyclodextrin. By exploiting the thermodynamics of the complexation in solution, a protective and stable environment can be formed for the guest, and this can be driven further by the addition of excess uncomplexed cyclodextrin. Release characteristics of a guest from the cylodextrin can be governed by K_{H}, the guest's air/water partition coefficient. K_{H} can be large compared to log(P) if the system comprising the cyclodextrin inclusion complex is placed in a non-equilibrium situation, such as the mouth. One of ordinary skill in the art will understand that more than one guest can be present in a system, and that similar equations and relationships can be applied to each guest of the system.

In embodiments in which the guest is a flavor and the commercial product is a beverage (or other liquid), the cyclodextrin can protect the flavor from degradation in the liquid product, but can release the flavor from encapsulation when the liquid is allowed to contact taste buds in the mouth. Thus, the desired flavor or essence of the product can be maintained, and the appropriate flavor or essence profile can be delivered, while preventing degradation of that flavor or essence, and while supplying a legally allowable amount of cyclodextrin to the beverage. This phenomenon is further described in Examples 21-22 and further illustrated in Tables 2 and 3 and FIGS. 7-10.

The direct comparison of beverages stabilized by either β-cyclodextrin or hydroxypropyl-β-cyclodextrin gave suprising and unexpected results. While hydroxypropyl-β-cyclodextrin may result in superior color stability (as shown in example 40); it is less effective than β-cyclodextrin in preventing flavor offnotes, even though its much greater water solubility was thought to enhance both modes of protection. Thus, the β cavity size and the unexpected low water solubility of β-cyclodextrin (1.85 g/100 ml), when compared to α-cyclodextrin (14.5 g/100 ml) and γ-cyclodextrin (23.2 g/100 ml), all measured at 25° C., seems to provide the thermodynamic environment necessary for nano-emulsion development and the stabilizing effects observed. Mixtures of β-cyclodextrin and hydroxypropyl-β-cyclodextrin may be used to gain both color stability and prevent offnotes. Suitably, β-cyclodextrin is present in from about 0.01 wt % to about 0.1 wt % in the finished product, while hydroxypropyl-β-cyclodextrin is present in from about 0.05 wt % to about 0.3 wt % in the finished product. Suitably, the hydroxypropyl-β-cyclodextrin and β-cyclodextrin are present in a ratio of from about 2:1 to about 1:30.

Various features and aspects of the invention are set forth in the following examples, which are intended to be illustrative and not limiting. All of the examples were performed at atmospheric pressure, unless stated otherwise.

### Example 1

### Cyclodextrin Inclusion Complex with β-Cyclodextrin and Diacetyl, Pectin as an Emulsifier, and Process for Forming Same

At atmospheric pressure, in a 100 gallon reactor, 49895.1600 g (110.02 lb) of β-cyclodextrin was dry blended with 997.9 g (2.20 lb) of beet pectin (2 wt % of pectin: β-cyclodextrin; XPQ EMP 5 beet pectin available from Degussa-France) to form a dry blend. The 100 gallon reactor was jacketed for heating and cooling, included a paddle agitator, and included a condenser unit. The reactor was supplied with a propylene glycol coolant at approximately 40° F. (4.5° C.). The propylene glycol coolant system is initially turned off, and the jacket acts somewhat as an insulator for the reactor. 124737.9 g (275.05 lb) of hot deionized water was added to the dry blend of β-cyclodextrin and pectin. The water had a temperature of approximately 118° F. (48° C.). The mixture was stirred for approximately 30 min. using the paddle agitator of the reactor. The reactor was then temporarily opened, and 11226.4110 g (24.75 lb) of diacetyl was added (as used hereinafter, “diacetyl” in the examples refers to diacetyl purchased from Aldrich Chemical, Milwaukee, Wis.). The reactor was resealed, and the resulting mixture was stirred for 8 hours with no added heat. Then, the reactor jacket was connected to the propylene glycol coolant system. The coolant was turned on to approximately 40° F. (4.5° C.), and the mixture was stirred for approximately 36 hours. The mixture was then emulsified using a high shear tank mixer, such as what is typically used in spray dry operations. The mixture was then spray dried on a nozzle dryer having an inlet temperature of approximately 410° F. (210° C.) and an outlet temperature of approximately 221° F. (105° C.). A percent retention of 12.59 wt % of diacetyl in the cyclodextrin inclusion complex was achieved. The moisture content was measured at 4.0%. The cyclodextrin inclusion complex included less than 0.3% surface diacetyl, and the particle size of the cyclodextrin inclusion complex was measured as 99.7% through an 80 mesh screen. Those skilled in the art will understand that heating and cooling can be controlled by other means. For example, diacetyl can be added to a room temperature slurry and can be automatically heated and cooled.

### Example 2

### Cyclodextrin Inclusion Complex with α-cyclodextrin and Diacetyl, Pectin as an Emulsifier, and Process for Forming Same

The β-cyclodextrin of example 1 was replaced with α-cyclodextrin and dry blended with 1 wt % pectin (i.e., 1 wt % of pectin: β-cyclodextrin; XPQ EMP 5 beet pectin available from Degussa-France). The mixture was processed and dried by the method set forth in Example 1. The percent retention of diacetyl in the cyclodextrin inclusion complex was 11.4 wt %.

### Example 3

### Cyclodextrin Inclusion Complex with β-cyclodextrin and Orange Essence, Pectin as an Emulsifier, and Process for Forming Same

Orange essence, an aqueous waste stream from juice production, was added as the aqueous phase to a dry blend of β-cyclodextrin and 2 wt % pectin, formed according to the process set forth in Example 1. No additional water was added, the solids content was approximately 28%. The cyclodextrin inclusion complex was formed by the method set forth in Example 1. The dry inclusion complex contained approximately 3 to 4 wt % acetaldehyde, approximately 5 to 7 wt % ethyl butyrate, approximately 2 to 3 wt % linalool and other citrus enhancing notes. The resulting cyclodextrin inclusion complex can be useful in top-noting beverages.

### Example 4

### Cyclodextrin Inclusion Complex WITH β-cyclodextrin and Acetyl Propionyl, Pectin as an Emulsifier, and Process for Forming Same

A molar excess of acetyl propionyl was added to a dry blend of β-cyclodextrin and 2 wt % pectin in water, following the method set forth in Example 1. The percent retention of acetyl propionyl in the cyclodextrin inclusion complex was 9.27 wt %. The mixture can be useful in top-noting diacetyl-free butter systems.

### Example 5

### Orange Oil Flavor Product and Process for forming Same

Orange oil (i.e., Orange Bresil; 75 g) was added to an aqueous phase comprising 635 g of water, 403.75 g of maltodextrin, and 21.25 g of beet pectin (available from Degussa—France, product no. XPQ EMP 5). The orange oil was added to the aqueous phase with gentle stirring, followed by strong stirring at 10,000 RPM to form a mixture. The mixture was then passed through a homogenizer at 250 bars to form an emulsion. The emulsion was dried using a NIRO-brand spray drier having an inlet temperature of approximately 180° C. and an outlet temperature of approximately 90° C. to form a dried product. The percent flavor retention was then quantified as the amount of oil (in g) in 100 g of the dried product, divided by the oil content in the starting mixture. The percent retention of orange oil was approximately 91.5%.

### Example 6

### Orange Oil Flavor Product and Process for Forming Same

Orange oil (75 g) was added to an aqueous phase comprising 635 g of water, 297.50 g of maltodextrin, and 127.50 g gum arabic (available from Colloids Naturels International). The orange oil was added to the aqueous phase and dried following the method set forth in Example 5. The percent flavor retention was approximately 91.5%.

### Example 7

### Orange Oil Flavor Product and Process for Forming Same

Orange oil (75 g) was added to an aqueous phase comprising 635 g of water, 297.50 g of maltodextrin, 123.25 g gum arabic (available from Colloids Naturels International), and 4.25 g of depolymerized citrus pectin. The orange oil was added to the aqueous phase and dried following the method set forth in Example 5. The percent flavor retention was approximately 96.9%.

### Example 8

### Orange Oil Flavor Product and Process for Forming Same

Orange oil (75 g) was added to an aqueous phase comprising 635 g of water, 297.50 g of maltodextrin, 123.25 g gum arabic (available from Colloids Naturels International), and 4.25 g of beet pectin (available from Degussa—France, product no. XPQ EMP 5). The orange oil was added to the aqueous phase and dried following the method set forth in Example 5. The percent flavor retention was approximately 99.0%.

### Example 9

### Orange Oil Flavor Product and Process for Forming Same

Orange oil (75 g) was added to an aqueous phase comprising 635 g of water, 403.75 g of maltodextrin, and 21.25 g of depolymerized citrus pectin. The orange oil was added to the aqueous phase and dried following the method set forth in Example 5. The percent flavor retention was approximately 90.0%.

### Example 10

### Orange Oil Flavor Product and Process for Forming Same

Orange oil (75 g) was added to an aqueous phase comprising 635 g of water, 340.00 g of maltodextrin, and 85.00 g gum arabic (available from Colloids Naturels International). The orange oil was added to the aqueous phase and dried following the method set forth in Example 5. The percent flavor retention was approximately 91.0%.

### Example 11

### Orange Oil Flavor Product and Process for Forming Same

Orange oil (75 g) was added to an aqueous phase comprising 635 g of water and 425.00 g of maltodextrin. The orange oil was added to the aqueous phase and dried following the method set forth in Example 5. The percent flavor retention was approximately 61.0%.

### Example 12

### Orange Oil Flavor Product and Process for Forming Same

Orange oil (75 g) was added to an aqueous phase comprising 635 g of water, 420.75 g of maltodextrin, and 4.25 g of pectin. The orange oil was added to the aqueous phase and dried following the method set forth in Example 5. The percent flavor retention was approximately 61.9%.

### Example 13

### Orange Oil Flavor Product and Process for Forming Same

Orange oil (75 g) was added to an aqueous phase comprising 635 g of water, 403.75 g of maltodextrin, and 21.50 g of pectin. The orange oil was added to the aqueous phase and dried following the method set forth in Example 5. The percent flavor retention was approximately 71.5%.

### Example 14

### Orange Oil Flavor Product and Process for Forming Same

Orange oil (75 g) was added to an aqueous phase comprising 635 g of water, 420.75 g of maltodextrin, and 4.75 g of depolymerized citrus pectin. The orange oil was added to the aqueous phase and dried following the method set forth in Example 5. The percent flavor retention was approximately 72.5%.

### Example 15

### Orange Oil Flavor Product and Process for Forming Same

Orange oil (75 g) was added to an aqueous phase comprising 635 g of water, 420.75 g of maltodextrin, and 4.75 g of beet pectin (available from Degussa-France, product no. XPQ EMP 5). The orange oil was added to the aqueous phase and dried following the method set forth in Example 5. The percent flavor retention was approximately 78.0%.

### Example 16

### Orange Oil Flavor Product and Process for Forming Same

Orange oil (75 g) was added to an aqueous phase comprising 635 g of water, 414.40 g of maltodextrin, and 10.60 g of depolymerized citrus pectin. The orange oil was added to the aqueous phase and dried following the method set forth in Example 5. The percent flavor retention was approximately 85.0%.

### Example 17

### Orange Oil Flavor Product and Process for Forming Same

Orange oil (75 g) was added to an aqueous phase comprising 635 g of water, 414.40 g of maltodextrin, and 10.60 g of beet pectin (available from Degussa-France, product no. XPQ EMP 5). The orange oil was added to the aqueous phase and dried following the method set forth in Example 5. The percent flavor retention was approximately 87.0%.

### Example 18

### Cyclodextrin Inclusion Complex with β-cyclodextrin and Citral, Pectin as an Emulsifier, and Process for Forming Same

At atmospheric pressure, in a 1-L reactor, 200 g of β-cyclodextrin was dry blended with 4.0 g of beet pectin (2 wt % of pectin: β-cyclodextrin; XPQ EMP 5 beet pectin available from Degussa-France) to form a dry blend. 500 g of deionized water was added to the dry blend of β-cyclodextrin and pectin to form a slurry or mixture. The 1-L reactor was set up for heating and cooling via a lab-scale water bath heating and cooling apparatus. The mixture was heated at 55-60 degrees C. for 5 hours and agitated by stirring. 27 g of citral (natural citral, SAP No. 921565, Lot No. 10000223137, available from Citrus & Allied) was added. The reactor was sealed, and the resulting mixture was stirred for 5 hours at about 55-60 degrees C. The cooling portion of the heating and cooling lab apparatus was then turned on, and the mixture was stirred overnight at about 5-10 degrees C. The mixture was then spray dried on a BUCHI B-191 lab spray dryer (available from Buchi, Switzerland) having an inlet temperature of approximately 210 degrees C. and an outlet temperature of approximately 105 degrees C. A percent retention of 11.5 wt % of citral in the cyclodextrin inclusion complex was achieved. The resulting dry powder included 0.08 wt % surface oils (free citral).

### Example 19A

### Flavor Composition Comprising Cyclodextrin-Encapsulated Citral and Excess Uncomplexed Cyclodextrin

Encapsulated citral was produced according to the method set forth in Example 18. The resulting dry powder including the cyclodextrin-encapsulated citral was dry blended with additional β-cyclodextrin to achieve a wt % of about 1.0 wt % of the complex of Example 18 or about 0.1 wt % of citral in the resulting dry powder mixture (“citral-cyclodextrin/cyclodextrin blend”). The citral-cyclodextrin/cyclodextrin blend was added to an acidic beverage in a wt % of about 0.2 wt % of the dry powder mixture (i.e., β-cyclodextrin-encapsulated citral plus additional β-cyclodextrin) to the total weight of the beverage. This provided 10-15 ppm of citral and about 0.2 wt of β-cyclodextrin to the acidic beverage.

### Example 19B

### Flavor Composition Comprising Cyclodextrin-Encapsulated Citral and Excess Uncomplexed Cyclodextrin

Encapsulated citral is produced according to the method set forth in Example 18. The resulting dry powder including the cyclodextrin-encapsulated citral is dry blended with additional β-cyclodextrin to achieve a wt % of about 0.1 wt % of citral in the resulting dry powder mixture (“citral-cyclodextrin/cyclodextrin blend”). The citral-cyclodextrin/cyclodextrin blend is added to a beverage as a topnote. The citral-cyclodextrin/cyclodextrin blend is added in a wt % of about 0.2 wt % of the dry powder mixture (i.e., β-cyclodextrin-encapsulated citral plus additional β-cyclodextrin) to the total weight of the beverage.

An additional dilution/dry blend of encapsulated citral set forth in Example 18 with additional excess β-cyclodextrin to achieve 0.2 wt % active citral. This added dilution is necessary to study the effect of 0.1% β-cyclodextrin in beverage while providing an identical level of citral.

### Example 19C

### Flavor Composition Comprising Cyclodextrin-Encapsulated Citral and Excess Uncomplexed Hydroxypropyl β-cyclodextrin

Encapsulated citral is produced according to the method set forth in Example 18. The resulting dry powder including the cyclodextrin-encapsulated citral was dry blended with hydroxypropyl β-cyclodextrin (Aldrich Chemical, Milwaukee Wis.) to achieve a wt % of about 0.1 wt % of citral in the resulting dry powder mixture (“citral-cyclodextrin/cyclodextrin blend”). The citral-cyclodextrin/cyclodextrin blend was added to an acidic beverage in a wt % of about 0.2 wt % of the dry powder mixture (i.e., β-cyclodextrin-encapsulated citral plus additional hydroxypropyl β-cyclodextrin) to the total weight of the beverage. This provided 10-15 ppm of citral and about 0.2 wt % of hydroxypropyl β-cyclodextrin.

An additional dilution/dry blend of encapsulated citral set forth in Example 18 with additional excess hydroxypropyl β-cyclodextrin to achieve 0.2 wt % active citral. This added dilution is necessary to study the effect of 0.1% hydroxypropyl β-cyclodextrin in beverage while providing an identical level of citral.

### Example 20

### Stabilization of Citral with Cyclodextrin

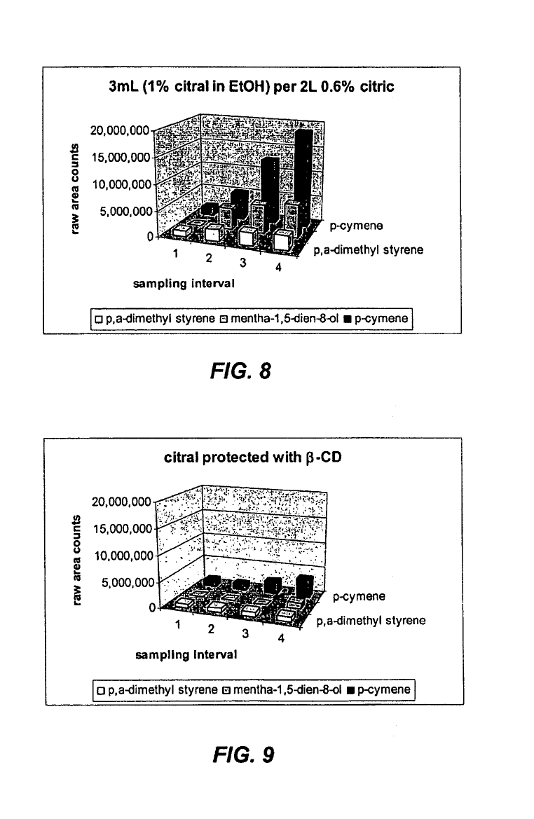

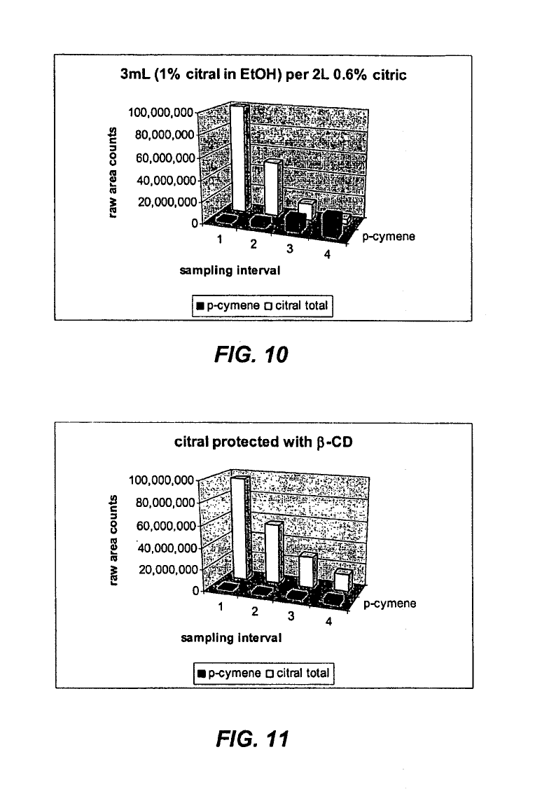

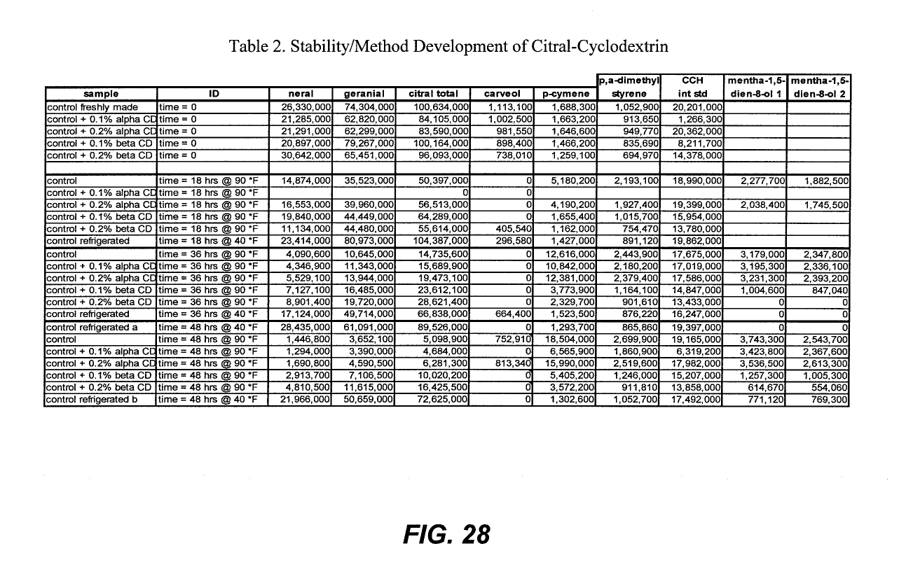

Citral (natural citral, SAP No. 921565, Lot No. 10000223137, available from Citrus & Allied) was cut in ethanol and diluted in citric acid to obtain a desired flavor level (e.g., 3 mL (1% citral in EtOH) per 2 L 0.6% citric acid; designated as “control” or “control freshly made” in Table 2). Then, 0.1 wt % and 0.2 wt % of α-cyclodextrin or β-cyclodextrin was added to the control and maintained at 40 degrees F. or 90 degrees F. for 18 hours, 36 hours, or 48 hours to simulate various shelf lives. The raw area counts of various forms of citral or character-impact citrus flavor compounds (i.e., neral, geranial, and citral total, the sum of neral and geranial), and a variety of other compounds, including common citrus flavor off-note chemicals (e.g., carveol, p-cymene or p-cymene-8-ol, p,a-dimethyl styrene, mentha-1,5-dien-8-ol 1, and mentha-a,5-dien-8-ol 2) and chlorocyclohexane internal standard (designated as “CCH int std” in Table 2) were measured for each permutation of the experiment, as shown in Table 2. As used herein, the term “raw area counts” is used to refer to the area under the curve of a corresponding portion of a gas chromatogram when the samples are analyzed using a gas chromatography-mass spectrometry analysis, namely, a PEGASUS II Time-of-flight mass spectrometer (TOF-MS; available from LECO Corp., St. Joseph, Mich.). The chlorocyclohexane internal standard was included at 10 ppm per beverage to attempt to normalize the raw area counts of the other compounds of interest. As shown in Table 2 (FIG. 28), the addition of cyclodextrin (and particularly, β-cyclodextrin) increased the amount of citral in the solution, and decreased the amount of off-notes formed. Specifically, this phenomenon was observed as simulated shelf-life increased (i.e., a greater distinction was observed between solutions containing cyclodextrin, and particularly, β-cyclodextrin and the control as time and temperature increased). This can be seen by comparing FIG. 8 and FIG. 9, which illustrate the inhibition of off-note formation with the addition of β-cyclodextrin. This can further be seen by comparing FIG. 10 and FIG. 11, which illustrate a sustained citral (and other character-impact citrus flavor) contribution to the beverage at later time intervals and lack of off-notes at later time intervals with the addition of β-cyclodextrin.

### Example 21

### Stability of Cyclodextrin-Encapsulated Citral in Acid

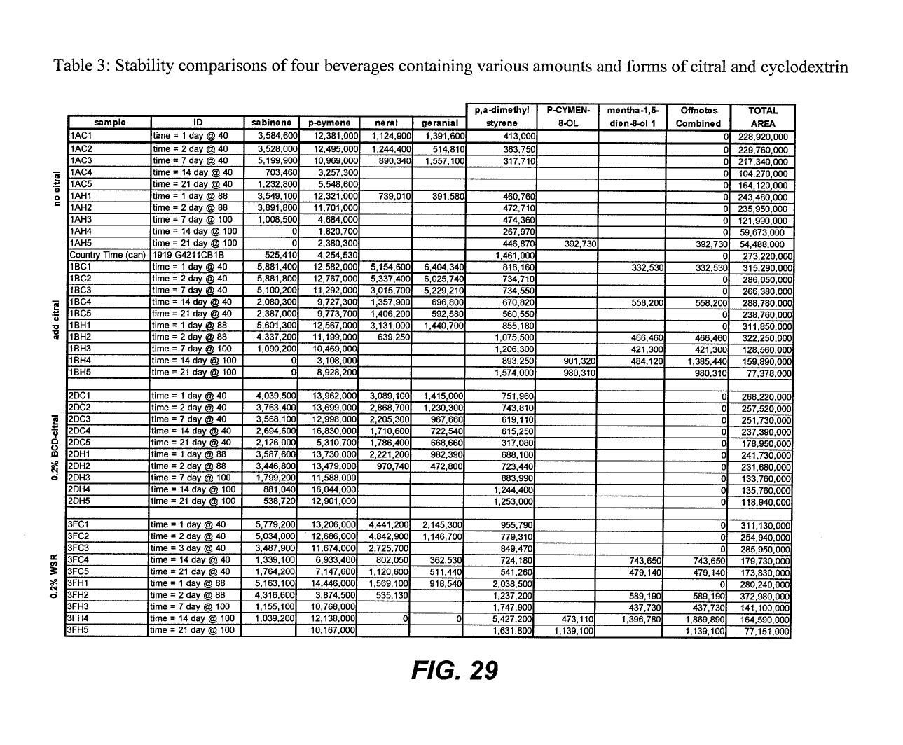

As shown in Table 3 (FIG. 29), four different versions of a sample acid beverage were analyzed. The four sample beverages were formed by adding various forms of citral to a low pH lemonade base, or an “acid-sugar” solution (e.g., 0.5% citric acid and 8% sugar in water). The first beverage, referred to in Table 3 as “no citral,” was formed by adding a non-citral citrus flavor component to the acid-sugar solution. The second beverage, “add citral,” was formed by adding 3 mL (1% citral in EtOH) per 2 L 0.6% citric acid (the citral used was natural citral, SAP No. 921565, Lot No. 10000223137, available from Citrus & Allied) to the acid-sugar solution to achieve a citral concentration of about 10-15 ppm. The third beverage, “0.2% BCD-citral,” was formed by adding 0.2 wt % of the citral-cyclodextrin/cyclodextrin blend formed in Example 19A to the acid-sugar solution to achieve a citral concentration of about 10-15 ppm. The fourth beverage, “0.2% WSR,” was formed by adding 0.2 wt % of water-soluble rosemary to the second beverage, while maintaining a citral concentration of about 10-15 ppm. Water soluble rosemary (“WSR”) as used herein refers to the industry standard used in stabilizing water-miscible flavorings.

The raw area counts of various forms of citral or character-impact citrus flavor compounds (i.e., sabinene, p-cymene, neral, and geranial), and a variety of other compounds, including common citral off-note chemicals (e.g., p,a-dimethyl styrene, p-cymene-8-ol, and mentha-1,5-dien-8-ol 1) were measured for each of the four beverages. Measurements were taken after 1 day at 40 degrees F., 1 day at 88 degrees F., 2 days at 40 degrees F., 2 days at 88 degrees F., 7 days at 40 degrees F., 7 days at 100 degrees F., 14 days at 40 degrees F., 14 days at 100 degrees F., 21 days at 40 degrees F., and 21 days at 100 degrees F. to simulate various shelf lives. In addition, the raw area counts of the above compounds in a can of Country Time®-brand lemonade were determined.

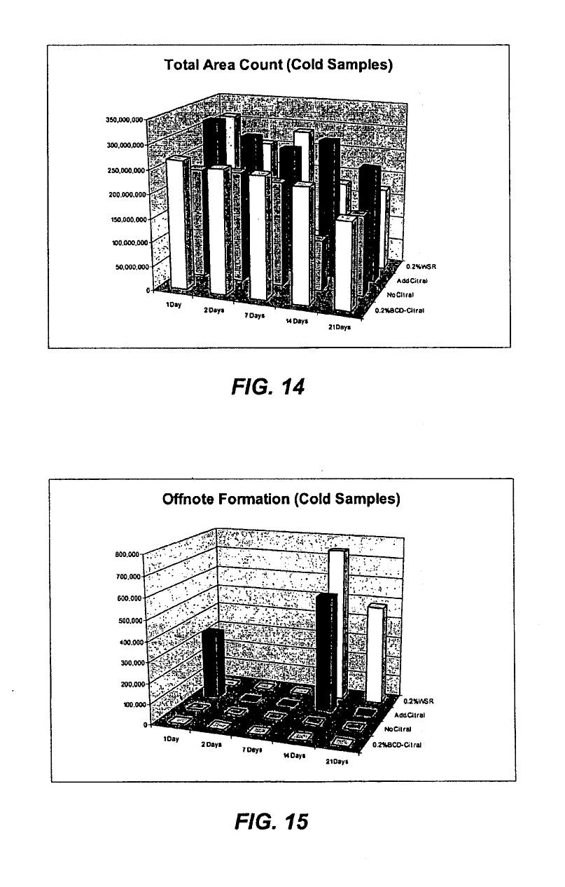

As shown in Table 3 and FIG. 12 and FIG. 13, at warmer temperatures (i.e., 88 degrees F. and 100 degrees F.), the third beverage included similar raw area counts of citral and other citrus flavor compounds as the other beverages (see FIG. 12), but with the lowest raw area counts of off-notes formed at all time intervals (see FIG. 13). As shown in FIGS. 14 and 15, at a colder temperature (i.e., 40 degrees F.), the third beverage included similar raw area counts of citral and other citrus flavor compounds as the other beverages (see FIG. 14), but with lower raw area counts of off-notes formed at all time intervals than the second and third beverages and the same raw area counts of off-notes formed in the first beverage to which no citral was added (see the “Offnotes Combined” column in Table 2 and FIG. 15).

As shown in Table 3 (FIG. 29), mentha-1,5-dien-8-ol is the first off-note to form from unprotected citral, which further degrades to p-cymen-8-ol over time. However, neither off-note was present in the third beverage, which includes the citral-cyclodextrin/cyclodextrin blend. Also, the 0.2% BCD-citral was better at stabilizing citral and other citrus flavor compounds than the industry standard WSR.

### Example 22

### Stability of Cyclodextrin-Encapsulated Citral in Acid

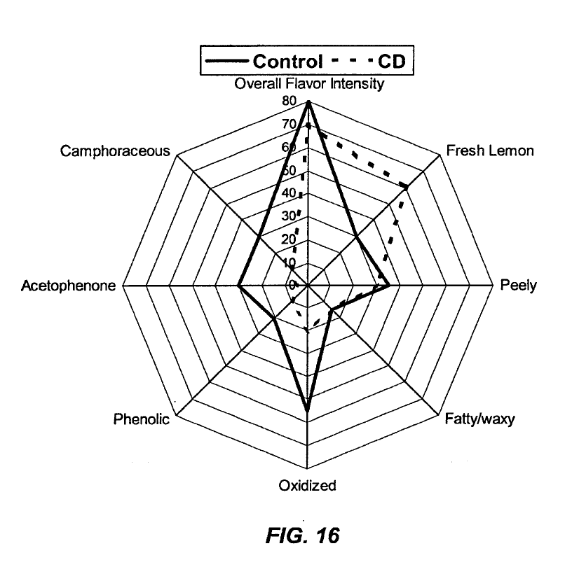

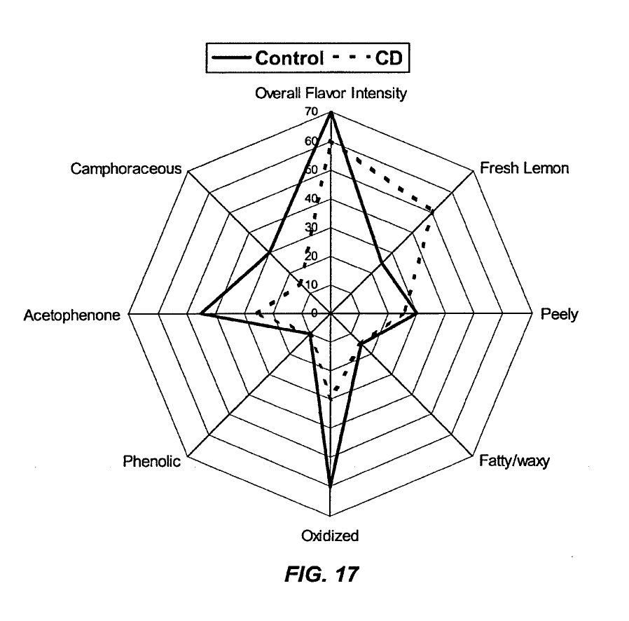

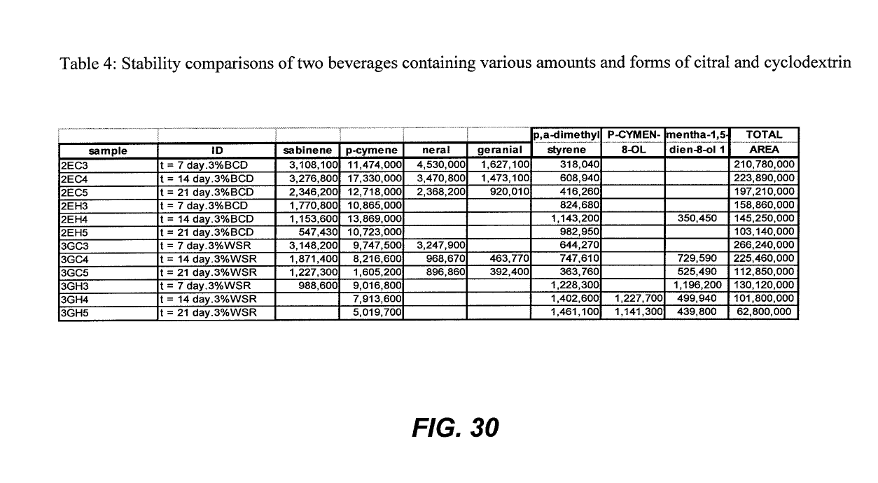

A first beverage, referred to as “0.3% BCD” in the ID column of Table 4, was formed by adding 0.3 wt % of the citral-cyclodextrin/cyclodextrin blend formed in Example 19A to the acid-sugar solution to achieve a citral concentration of about 20 ppm. A second beverage, “0.3% WSR,” was formed by adding 0.3 wt % of WSR to the second beverage of Example 21, while maintaining a citral concentration of about 10-15 ppm. The raw area counts of various forms of citral or citrus flavor compounds (i.e., sabinene, p-cymene, neral, and geranial), and a variety of other compounds, including common citral off-note chemicals (e.g., p,a-dimethyl styrene, p-cymene-8-ol, and mentha-1,5-dien-8-ol 1) were measured for each of the two beverages. Measurements were taken after 7 days at 40 degrees F., 7 days at 100 degrees F., 14 days at 40 degrees F., 14 days at 100 degrees F., 21 days at 40 degrees F. and 21 days at 100 degrees F. to simulate various shelf lives. As shown in Table 4 (FIG. 30), at the warmer temperature and the colder temperature, the first beverage included similar maintenance of citral (and other character-impact citrus flavor) contribution as the other beverage, but enhanced inhibition of the formation of off-notes at all time intervals. A general decrease in volatiles was noted due to interactions with the beverage container. However, the very strong complexes that formed between citral and β-cyclodextrin may be partially responsible for the reduction in headspace values for citral. Citral is, nevertheless, available for taste, as shown in the sensory analyses (Example 34 and FIGS. 16 and 17), and as previously described.

### Example 23

### Cyclodextrin Inclusion Complex with β-cyclodextrin and Lemon Oil 3X, Pectin as an Emulsifier, and Process for Forming Same

At atmospheric pressure, in a 1-L reactor, 400 g of β-cyclodextrin was dry blended with 8.0 g of beet pectin (2 wt % of pectin: β-cyclodextrin; XPQ EMP 5 beet pectin available from Degussa-France) to form a dry blend. 1 L of deionized water was added to the dry blend of β-cyclodextrin and pectin to form a slurry or mixture. The 1-L reactor was set up for heating and cooling via a lab-scale water bath heating and cooling apparatus. The mixture was stirred for about 30 min. 21 g of 3X (i.e., 3-fold) California Lemon Oil, available from Citrus & Allied) was added. The reactor was sealed, and the resulting mixture was stirred for 4 hours at about 55-60 degrees C. The cooling portion of the heating and cooling lab apparatus was then turned on, and the mixture was stirred overnight at about 5-10 degrees C. The mixture was then spray dried on a BUCHI B-191 lab spray dryer (available from Buchi, Switzerland) having an inlet temperature of approximately 210 degrees C. and an outlet temperature of approximately 105 degrees C. A percent retention of 4.99 wt % of lemon oil 3X in the cyclodextrin inclusion complex was achieved.

### Example 24A

### Flavor Composition Comprising Cyclodextrin-Encapsulated Lemon Oil 3X and Excess Uncomplexed Cyclodextrin Used in Beverage Product

The dry powder resulting from Example 23 including the cyclodextrin-encapsulated lemon oil 3X is dry blended with additional β-cyclodextrin to achieve a wt % of about 1 wt % of lemon oil 3X in the resulting dry powder mixture (“lemon oil 3X-cyclodextrin/cyclodextrin blend”). The lemon oil 3X-cyclodextrin/cyclodextrin blend is then added to a beverage in a wt % ranging from about 0.05 wt % to about 0.30 wt % of the dry powder mixture (i.e., β-cyclodextrin-encapsulated citral plus additional β-cyclodextrin) to the total weight of the beverage. This is expected to provide 20-30 ppm of lemon oil 3X and from about 0.05 wt % to about 0.30 wt % of β-cyclodextrin to the beverage, depending on the amount of dry powder mixture added to the beverage.

### Example 24B

### Flavor Composition Comprising Cyclodextrin-Encapsulated Lemon Oil 3X and Excess Uncomplexed Cyclodextrin Used in Beverage Product

The combination of the dry powder from Example 24 mixed with the citral-cyclodextrin inclusion complex from Example 18 is blended (5 parts citral/3 parts 3X lemon) and blended with additional β-cyclodextrin to achieve a 1% active flavor in cyclodextrin. The mixture is useful in delivering a stable peely, fresh lemon character in spices and condiments with a high acid content (acetic) or in beverage where a more opaque, juice like appearance is desired, with high stability.

### Example 25

### Cyclodextrin Inclusion Complex with β-cyclodextrin and Alpha-Tocopherol, Pectin as an Emulsifier, and Process for Forming Same

At atmospheric pressure, in a 1-L reactor, 200 g of β-cyclodextrin was dry blended with 4.0 g of beet pectin (2 wt % of pectin: β-cyclodextrin; XPQ EMP 5 beet pectin available from Degussa-France) to form a dry blend. 500 g of deionized water was added to the dry blend of β-cyclodextrin and pectin to form a slurry or mixture. The 1-L reactor was set up for heating and cooling via a lab-scale water bath heating and cooling apparatus. The mixture was stirred for about 30 min. 23 g of D,L-alpha-tocopherol (Kosher, SAP# 1020477, available from BASF) was added. The reactor was sealed, and the resulting mixture was stirred overnight at about 55-60 degrees C. The cooling portion of the heating and cooling lab apparatus was then turned on, and the mixture was stirred overnight at about 5-10 degrees C. The mixture was then spray dried on a BUCHI B-191 lab spray dryer (available from Buchi, Switzerland) having an inlet temperature of approximately 210 degrees C. and an outlet temperature of approximately 105 degrees C. A percent retention of 10.31 wt % of alpha-tocopherol in the cyclodextrin inclusion complex was achieved. A 1:1 mole ratio of alpha tocopherol in β-cyclodextrin would correspond to 27.52 wt %, however, the literature reports this to be an oily paste. The 10.31 wt % product is a dry, free flowing powder that can easily be dispersed in water. The 10.31 wt % alpha tocopherol complex easily disperses in water when used at 0.1% (i.e., cut in excess uncomplexed β-cyclodextrin).

### Example 26

### Composition Comprising Cyclodextrin-Encapsulated Alpha-Tocopherol and Excess Uncomplexed Cyclodextrin Used in Beverage Product

The dry powder resulting from Example 25 that includes the cyclodextrin-encapsulated alpha-tocopherol is dry blended with additional β-cyclodextrin to achieve a wt % of about 1 wt % of alpha-tocopherol in the resulting dry powder mixture (“alpha-tocopherol-cyclodextrin/cyclodextrin blend”). The alpha-tocopherol-cyclodextrin/cyclodextrin blend is then added to a beverage as an antioxidant and/or a nutraceutical to an A.C.E. beverage (i.e., A=vitamin A, C=vitamin C, and E=vitamin E) in a wt % of about 0.2 wt % of the dry powder mixture (i.e., β-cyclodextrin-encapsulated alpha-tocopherol plus additional β-cyclodextrin) to the total weight of the beverage. This is expected to provide 10 ppm of alpha-tocopherol and about 0.2 wt % of β-cyclodextrin to the acidic beverage.

### Example 27

### Flavor Composition Comprising Cyclodextrin-Encapsulated Alpha-Tocopherol and Excess Uncomplexed Cyclodextrin Used in Beverage Product

The dry powder resulting from Example 25 including the cyclodextrin-encapsulated alpha-tocopherol is combined with other flavor compositions (e.g., the citral-β-cyclodextrin formed according to Example 18, and/or the lemon oil 3X-β-cyclodextrin formed according to Example 23) and then dry blended with additional β-cyclodextrin to achieve the desired level of flavor components and alpha-tocopherol in the resulting dry powder mixture. The resulting dry powder mixture is then added to a beverage as an antioxidant/nutraceutical/flavor composition. This is expected to deliver the appropriate amount of antioxidant/nutraceutical and flavor profile to the beverage, and an appropriate amount of β-cyclodextrin to the beverage (e.g., 0.2 wt %). In beverages, such a combination is expected to provide flavor, cloud (i.e., juice-like appearance), added stability to citrus components, and demonstrates the advantage of being able to blend flavor level, cloud and functionality. It is anticipated that such a system is highly effective in salad dressing and seasoning mixes, at least partially because of the enhanced citrus protection coupled with added lipid protection.

### Example 28

### Cyclodextrin Inclusion Complex with β-cyclodextrin and Lemon Lime Oils, Pectin as an Emulsifier and Xanthan Gum as a Thickener, and Process for Forming Same

In a 1-L reactor, 400 g of β-cyclodextrin (W713-cyclodextrin, available from Wacker), 8 g of beet pectin (2 wt % of pectin: β-cyclodextrin; XPQ EMP 4 beet pectin available from Degussa-France), and 1.23 g xanthan gum (KELTROL xanthan gum, available from CP Kelco SAP No. 15695) were dry blended together to form a dry blend. 800 mL of deionized water were added to the dry blend to form a slurry or mixture. The 1-L reactor was set up for heating and cooling via a lab-scale water bath heating and cooling apparatus. The mixture was agitated by stirring for about 30 min. 21 g of lemon lime flavor 043-03000 (SAP# 1106890, available from Degussa Flavors & Fruit Systems), were added. The reactor was sealed, and the resulting mixture was stirred for 4 hours at about 55-60 degrees C. The cooling portion of the heating and cooling lab apparatus was then turned on, and the mixture was stirred overnight at about 5-10 degrees C. The mixture was then spray dried on a BUCHI B-191 lab spray dryer (available from Buchi, Switzerland) having an inlet temperature of approximately 210 degrees C. and an outlet temperature of approximately 105 degrees C. A percent retention of about 4.99 wt % of lemon lime oils in the cyclodextrin inclusion complex was achieved.

### Example 29

### Cyclodextrin Inclusion Complex with β-cyclodextrin and Lemon Lime Oils, Pectin as an Emulsifier and Xanthan Gum as a Thickener, and Process for Forming Same

In a 1-L reactor, 300 g of β-cyclodextrin (W7 β-cyclodextrin, available from Wacker), 6 g of beet pectin (2 wt % of pectin: β-cyclodextrin; XPQ EMP 4 beet pectin available from Degussa-France), and 1.07 g xanthan gum (KELTROL xanthan gum, available from CP Kelco SAP No. 15695) were dry blended together to form a dry blend. 750 mL of deionized water were added to the dry blend to form a slurry or mixture. The 1-L reactor was set up for heating and cooling via a lab-scale water bath heating and cooling apparatus. The mixture was agitated by stirring for about 30 min. 16 g of lemon lime flavor 043-03000 (SAP# 1106890, available from Degussa Flavors & Fruit Systems), were added. The reactor was sealed, and the resulting mixture was stirred for 4 hours at about 55-60 degrees C. The cooling portion of the heating and cooling lab apparatus was then turned on, and the mixture was stirred overnight at about 5-10 degrees C. The mixture was then emulsified using a high shear tank mixer (HP 5 1PQ mixer, available from Silverston Machines Ltd., Chesham England). A percent retention of about 5.06 wt % of lemon lime oils in the cyclodextrin inclusion complex was achieved.

### Example 30

### Flavor Composition Comprising Cyclodextrin-Encapsulated Lemon Lime Oils and Excess Uncomplexed Cyclodextrin Used in Beverage Product

The dry powder resulting from Example 28, and/or the emulsion resulting from Example 29 including the cyclodextrin-encapsulated lemon lime oils is dry blended with additional β-cyclodextrin to achieve a wt % of about 1 wt % of lemon lime oils in the resulting dry powder mixture (“lemon lime oils-cyclodextrin/cyclodextrin blend”). The lemon lime oils-cyclodextrin/cyclodextrin blend is then added to a beverage in a wt % ranging from about 0.05 wt % to about 0.30 wt % of the dry powder mixture (i.e., β-cyclodextrin-encapsulated lemon lime oils plus additional β-cyclodextrin) to the total weight of the beverage. This is expected to provide 50-100 ppm of lemon lime oils and from about 0.05 wt % to about 0.30 wt % of β-cyclodextrin to the beverage, depending on the amount of dry powder mixture added to the beverage.

### Example 31

### Cyclodextrin Inclusion Complex with β-cyclodextrin and Citral, Pectin as an Emulsifier and Xanthan Gum as a Thickener, and Process for Forming Same

In a 1-L reactor, 300 g of β-cyclodextrin (W7 β-cyclodextrin, available from Wacker), 6 g of beet pectin (2 wt % of pectin: β-cyclodextrin; XPQ EMP 4 beet pectin available from Degussa-France), and 0.90 g xanthan gum (KELTROL xanthan gum, available from CP Kelco SAP No. 15695) were dry blended together to form a dry blend. 575 mL of deionized water were added to the dry blend to form a slurry or mixture. The 1-L reactor was set up for heating and cooling via a lab-scale water bath heating and cooling apparatus. The mixture was agitated by stirring for about 30 min. 18 g of citral (natural citral, SAP No. 921565, Lot No. 10000223137, available from Citrus & Allied), were added. The reactor was sealed, and the resulting mixture was stirred for 4 hours at about 55-60 degrees C. The cooling portion of the heating and cooling lab apparatus was then turned on, and the mixture was stirred over the weekend at about 5-10 degrees C. The mixture was then divided into two halves. One half was emulsified neat using a high shear tank mixer (HP 5 1PQ mixer, available from Silverston Machines Ltd., Chesham England). 1 wt % gum acacia was added to the other half, and the resulting mixture was emulsified using the same high shear tank mixer. A percent retention of about 2.00 wt % of citral in the cyclodextrin inclusion complex was achieved.

### Example 32

### Flavor Emulsion Comprising Cyclodextrin-Encapsulated Citral Used in Food or Beverage Product

One or both of the resulting emulsions from Example 31 including the cyclodextrin-encapsulated citral is added directly to a food or beverage product to obtain a stable product with the appropriate flavor profile. The emulsions are added directly to a food or beverage product, or sprayed onto a food substrate.

### Example 33

### Flavor Emulsion Comprising Cyclodextrin-Encapsulated Citral and Excess Uncomplexed Cyclodextrin Used in a Beverage Product

One (or a mixture of both) of the resulting emulsions formed according to Example 31 including the cyclodextrin-encapsulated citral is combined with additional cyclodextrin to achieve a wt % of about 1 wt % of citral in the resulting flavor emulsion (“citral-cyclodextrin/cyclodextrin emulsion”). The citral-cyclodextrin/cyclodextrin emulsion is added to a beverage in a wt % ranging from about 0.05 wt % to about 0.30 wt % of the flavor emulsion (i.e., β-cyclodextrin-encapsulated citral plus additional β-cyclodextrin) to the total weight of the beverage. This is expected to provide 10-20 ppm of citral and from about 0.05 wt % to about 0.30 wt % of β-cyclodextrin to the beverage, depending on the amount of flavor emulsion added to the beverage. One of ordinary skill in the art will recognize that the excess uncomplexed β-cyclodextrin need not first be added to the flavor emulsion, but rather the excess uncomplexed β-cyclodextrin and a flavor emulsion formed according to Example 31 can be added simultaneously to a beverage product.

### Example 34

### Sensory Analysis of Lemonade Beverage Comprising Cyclodextrin-Encapsulated Citral VS. Control Lemonade Beverage

Encapsulated citral was produced according to the method set forth in Example 18. The resulting dry powder including the cyclodextrin-encapsulated citral was dry blended with additional β-cyclodextrin to achieve a wt % of about 1 wt % of citral in the resulting dry powder mixture (“citral-cyclodextrin/cyclodextrin blend”). The citral-cyclodextrin/cyclodextrin blend then blended with standard spray-dried lemon oil flavor 073-00531 (32.0 parts) (Degussa Flavors & Fruit Systems) to form a flavor composition. The flavor composition was added to a lemonade beverage base in a wt % of about 0.2 wt % of the dry powder mixture (i.e., β-cyclodextrin-encapsulated citral plus additional β-cyclodextrin) to the total weight of the beverage. The lemonade beverage base included 10.5 g of the flavor composition, 0.54 g of sugar, 0.04 g of citric acid, 0.13 g of sodium benzoate, and 88.79 g water. This provided 10 ppm of citral and about 0.2 wt % of β-cyclodextrin to the acidic beverage. This beverage was identified as “CD” for the sensory analysis illustrated in FIGS. 16 and 17.

A first control flavor composition was prepared by combining a spray-dried citral (natural citral, SAP No. 921565, Lot No. 10000223137, available from Citrus & Allied) and spray-dried lemon oil flavor 073-00531 (32.0 parts) (Degussa Flavors & Fruit Systems). The spray-dried forms of the flavors were prepared according to standard spray-dry procedures known to those of ordinary skill in the art. The first control flavor composition was added to the same lemonade base beverage as described above to create a first control lemonade beverage having a citral flavor level of 10 ppm. The results of the sensory analysis comparing the first control lemonade beverage with the CD beverage are shown in FIG. 16. The sensory analysis was performed after the beverages had been stored in the dark at 110 degrees F. for 3 weeks to simulate an aged beverage. The sensory analysis was a descriptive analysis performed by a trained sensory panel of six expert tasters, using a consensus approach and reference standards. As shown in FIG. 16, the CD beverage had a similar overall flavor intensity, a similar peely flavor, a higher fresh lemon flavor, and a lower fatty/waxy, oxidized, phenolic, acetophenone and camphoraceous flavor than the first control lemonade beverage. This sensory analysis illustrates the ability of cyclodextrin in stabilizing the key note flavor, citral, and in preventing the formation of off-note flavors that detract from and diminish the fresh lemon flavor of a lemonade beverage.

A second control flavor composition was prepared by combining an emulsion of citral (natural citral, SAP No. 921565, Lot No. 10000223137, available from Citrus & Allied) and lemon oil flavor 073-00531 (Degussa Flavors & Fruit Systems). The emulsion was prepared according to standard emulsifying procedures known to those of ordinary skill in the art. The second control flavor composition was added to the same lemonade base beverage as described above to create a second control lemonade beverage having a citral flavor level of 10 ppm. The results of the sensory analysis comparing the second control lemonade beverage with the CD beverage are shown in FIG. 17. The sensory analysis was performed after the beverages had been stored in the dark at 110 degrees F. for 3 weeks to simulate an aged beverage. The sensory analysis was a descriptive analysis performed by a trained sensory panel of six expert tasters, using a consensus approach and reference standards. As shown in FIG. 17, the CD beverage had a similar overall flavor intensity, a similar peely flavor, a higher fresh lemon flavor, and a lower fatty/waxy, oxidized, phenolic, acetophenone and camphoraceous flavor than the second control lemonade beverage. This sensory analysis illustrates the ability of cyclodextrin in stabilizing the key note flavor, citral, and in preventing the formation of off-note flavors that detract from and diminish the fresh lemon flavor of a lemonade beverage. As illustrated by comparing FIGS. 16 and 17, the second control lemonade beverage had higher perceived levels of oxidized and acetophenone flavors than the first control lemonade beverage. This could be because the second control flavor composition was in a liquid form, which could have led to a more accelerated degradation of key note flavors and off-note formation.

### Example 35

### Cyclodextrin Inclusion Complex with β-cyclodextrin and Citral, Pectin as an Emulsifier and Xanthan Gum as a Thickener, and Process for Forming Same

In a 5-L reactor a base formula of 86.25 g of β-cyclodextrin (W7 β-cyclodextrin, available from Wacker), 1.70 g of beet pectin (2 wt % of pectin: β-cyclodextrin; XPQ EMP 4 beet pectin available from Degussa-France), and 0.35 g xanthan gum (KELTROL xanthan gum, available from CP Kelco SAP No. 15695) were dry blended together to form a dry blend. 216.50 mL of deionized water were added to the dry blend to form a slurry or mixture. The 5-L reactor was set up for heating and cooling via a lab-scale water bath heating and cooling apparatus. The mixture was stirred for about 30 min. 11.7 g of citral (natural citral, SAP No. 921565, Lot No. 10000223137, available from Citrus & Allied) were added. This base formulation was scaled to produce 2200 g. The reactor was sealed, and the resulting mixture was stirred for 4 hours at about 55-60 degrees C. The cooling portion of the heating and cooling lab apparatus was then turned on, and the mixture was stirred overnight at about 5-10 degrees C. The mixture was then spray dried on a Niro Basic Lab Dryer (Niro Corp. Columbia, Md.) having an inlet temperature of approximately 210 degrees C. and an outlet temperature of approximately 105 degrees C. A percent retention of about 11.5 wt % of citral in the cyclodextrin inclusion complex was achieved.

### Example 36

### Cyclodextrin Inclusion Complex with β-cyclodextrin and Lemon Oil 3X, Pectin as an Emulsifier and Xanthan Gum as a Thickener, and Process for Forming Same

In a 5-L reactor, a base formulation of 92.95 g of β-cyclodextrin (W7 β-cyclodextrin, available from Wacker), 1.8 g of beet pectin (2 wt % of pectin: β-cyclodextrin; XPQ EMP 4 beet pectin available from Degussa-France), and 0.35 g xanthan gum (KELTROL xanthan gum, available from CP Kelco SAP No. 15695) were dry blended together to form a dry blend. 235.00 mL of deionized water were added to the dry blend to form a slurry or mixture. The 5-L reactor was set up for heating and cooling via a lab-scale water bath heating and cooling apparatus. The mixture was stirred for about 30 min. 4.9 g of 3X California lemon oil (available from Citrus & Allied) were added. The base formula was scaled up to produce 2200 g of product. The reactor was sealed, and the resulting mixture was stirred for 4 hours at about 55-60 degrees C. The cooling portion of the heating and cooling lab apparatus was then turned on, and the mixture was stirred overnight at about 5-10 degrees C. The mixture was then spray dried on a on a Niro Basic Lab Dryer (Niro Corp. Columbia, Md.) having an inlet temperature of approximately 210 degrees C. and an outlet temperature of approximately 105 degrees C. A percent retention of about 5 wt % of lemon oil 3X in the cyclodextrin inclusion complex was achieved.

### Example 37

### Off-Note Formation Comparison of Lemonade Beverage Comprising Cyclodextrin-Encapsulated Citral, Cyclodextrin-Encapsulated Lemon Oil 3X, and Excess Uncomplexed Cyclodextrin VS. a Cyclodextrin-Free Control Beverage

A lemonade base was prepared by combining 89.79 g water, 9.42 g of granulated sugar, 0.04 g of finely granulated sodium citrate, and 0.50 g of citric acid (anhydrous, fine). A preservative was not added to the beverage, but the beverage was subjected to a pasteurization hot pack. This base was scaled to produce 8 L finished beverage.

A beverage identified as “CD” was formed comprising a citral-cyclodextrin inclusion complex formed according to Example 35 (“citral-CD”) and a lemon oil 3X-cyclodextrin inclusion complex formed according to Example 36 (“lemon-CD”). A “CD” flavor composition was prepared by dry blending 32.00 g of spray-dried lemon oil (073-00531 available from Degussa Flavors & Fruit System), 5.20 g of citral-CD (073-00339 available from Degussa Flavors & Fruit System), 3.20 g of lemon-CD, and 59.60 g of excess uncomplexed β-cyclodextrin (W7 β-cyclodextrin, available from Wacker). The CD flavor composition was blended until uniform and screened using an approximately 30-mesh screen. The CD beverage was then prepared by adding 0.25 g of the CD flavor composition to the lemonade base and packaged in P.E.T. containers.

A control flavor composition was prepared by dry blending 32.00 g of spray-dried lemon oil, 5.20 g of spray-dried citral, and 3.20 g of spray-dried lemon oil 3X with 59.60 g of maltodextrin (all sprayed on maltodextrin (SAP No. 15433 available from Tate & Lyle). Each of the spray-dried flavors were spray-dried with maltodextrin according to standard spray-drying procedures known to those of ordinary skill in the art. The control flavor composition was completely free of cyclodextrin. A control beverage (referred to as “Unprotected”) was prepared by adding 0.25 g of the control flavor composition to the lemonade base and packaged in P.E.T. containers.

The flavor retention and off-note formation of the CD beverage was compared to that of the control beverage. The amount of citral and off-notes were determined using Solid Phase Micro Extraction (SPME), which is an analytical headspace technique that allows a high degree of automation and sensitivity with minimal sample preparation time. SPME has the same sub parts-per-million sensitivity as liquid-liquid extraction and distillation techniques but does not expose the sample to temperature extremes or use large amounts of solvents that can add contaminants and which need to be removed before analysis. SPME uses a 2 cm fiber assembly coated with a polymer (50/30 μm DVB/Carboxen™/PDMS StableFlex™—available from Supelco, Bellefonte, Pa.). The analytical sample is placed in a 10 mL crimp-top vial. By exposing the fiber to the headspace, which exists above the analytical sample, the organics are trapped in the polymer until thermally desorbed into the injection port of a gas chromatograph (GC) or GC-Mass Spectrometer (a PEGASUS III Time-of-flight mass spectrometer was used in this study (GC/TOF-MS; available from LECO Corp., St. Joseph, Mich.). The GC was an Agilent 6890 and the analysis performed on a 60 meter—x-0.32 mm—DB-5 column with a 1 micron film thickness (available from Restek Bellefonte, Pa.). Concentration effects on the order of 100,000 to 1,000,000 are easily obtained. In this study, 2 mL of each sample were placed in a 10 mL vial, which was thermostated at 50 degrees C. for 10 min. and extracted for 15 min. to obtain sub-parts-per-million sensitivity.

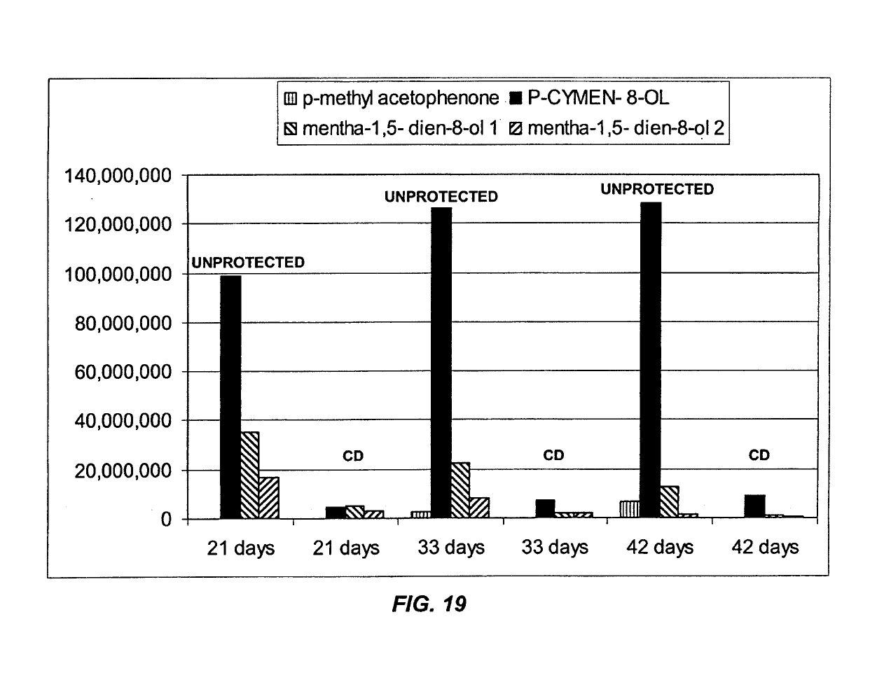

Flavor retention and total off-note growth at 88 degrees F. is shown for the Unprotected beverage and the CD beverage in FIG. 18. (The lighter bar represents the total flavor profile (i.e., all flavor components detected), and the darker bar represents the total off-note growth for the Unprotected beverage and the CD beverage.) As shown in FIG. 18, the CD beverage retained a significantly larger flavor profile longer than the Unprotected beverage, and the CD beverage had observably lower total off-note formation than the Unprotected beverage. Flavor migration into package materials is well documented in the literature and commercial information from package design firms. The prevention or mediation of flavor migration is significant and an un-expected benefit in addition to flavor stability. The formation of four types of off-notes were measured over time (i.e., after 21 days of storage at 88 degrees F., after 33 days of storage at 88 degrees F., and after 42 days of storage at 88 degrees F. in both beverages, and the results are shown in FIG. 19. Namely, the four off-notes that were analyzed were p-methyl acetophenone, p-cymen-8-ol, mentha-1,5-dien-8-ol 1 and mentha-1,5-dien-8-ol 2. As shown in FIG. 19, the CD beverage formed lower levels of all four off-notes than the Unprotected beverage, and particularly, formed lower levels of p-cymen-8-ol than the Unprotected beverage. The choice of off-notes followed is from the scheme detailed in FIG. 7. P-cresol, however, is very difficult to detect by SPME due to its high water solubility, thus p-cresol is characterized by a sensory panel. See, for example, FIGS. 16 and 17, where p-cresol is included as a phenolic note/attribute.

### Example 38

### Protective Effects in “Sun-Struck” Phenomenon Offered by β-cyclodextrin

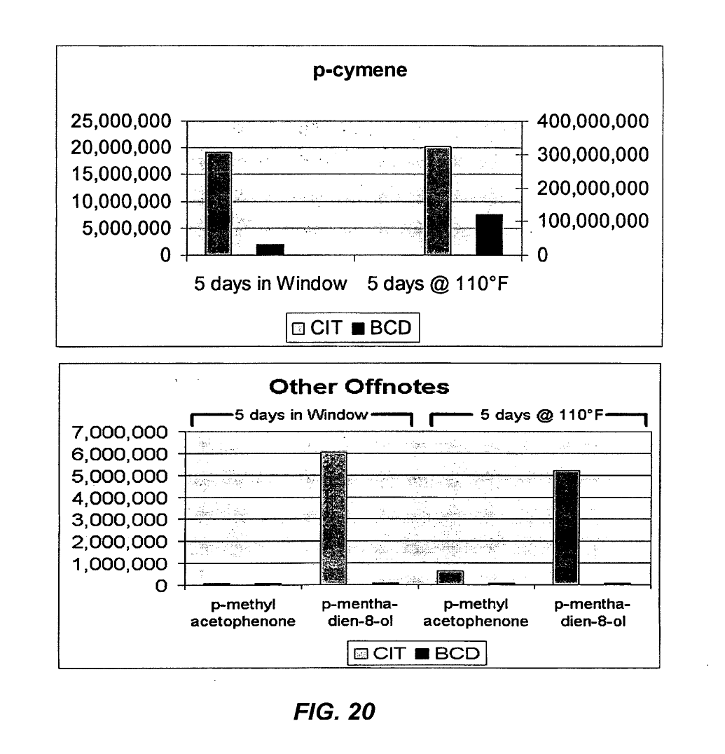

To study other protective effects offered by the incorporation of cyclodextrins into beverage products preliminary studies into the “Sun-Struck” (photooxidation) phenomenon were undertaken. Specifically, the sun exposure experienced by commercial products was studied. As in EXAMPLE 20, citral (natural citral, SAP No. 921565, available from Citrus & Allied) was diluted in ethanol at a level of 1.0%. Two simulated beverage bases were made: control, 0.6% citric acid in water and protected, 0.6% citric acid and 0.2% β-cyclodextrin in water. The 1.0% citral in ethanol solution was added to each beverage base at 0.1% (10 ppm citral); both simulated beverages were in glass juice bottles and placed in a lab window with south-east exposure that experiences strong sunlight for 5 days. Duplicate bottles of each simulated beverage were placed in an oven and maintained at 110 degrees F. After 5 days each bottle was sampled and analyzed by the same headspace methods employed throughout this research (SPME). The results are shown graphically in FIG. 20. Very little information is available on citral photo-stability, however, an examination of the offnotes in the un-protected sample shows very similar compounds and concentrations. It is, therefore, assumed that a similar reaction pathway is active in thermal and photo catalyzed degradation in acidic media (see, e.g., FIG. 7). In FIG. 20, the protected sample (labeled BCD) shows no formation of the reactive intermediate offnote p-mentha-dien-8-ol compared to the un-protected (labeled CIT). It is also evident that the formation of p-cymene is much reduced in the protected system.

### Example 39

### Stabilization of Citral, Color and Vitamin Content with Cyclodextrin

To a commercial vitamin fortified beverage (GLAC\check{E}AU multi-v lemonade (a-zinc) purchased at a local grocery store is added 0.2 wt % of the “citral-cyclodextrin/β-cyclodextrin blend” from Example 18 and 0.01 wt % red 40 color. The mixture is returned to the original container and sealed. The sealed bottle is place in a south facing window for 5-6 weeks or until color changes are noted.

### Example 40

### Stabilization of Citral, Color and Vitamin Content with Cyclodextrin

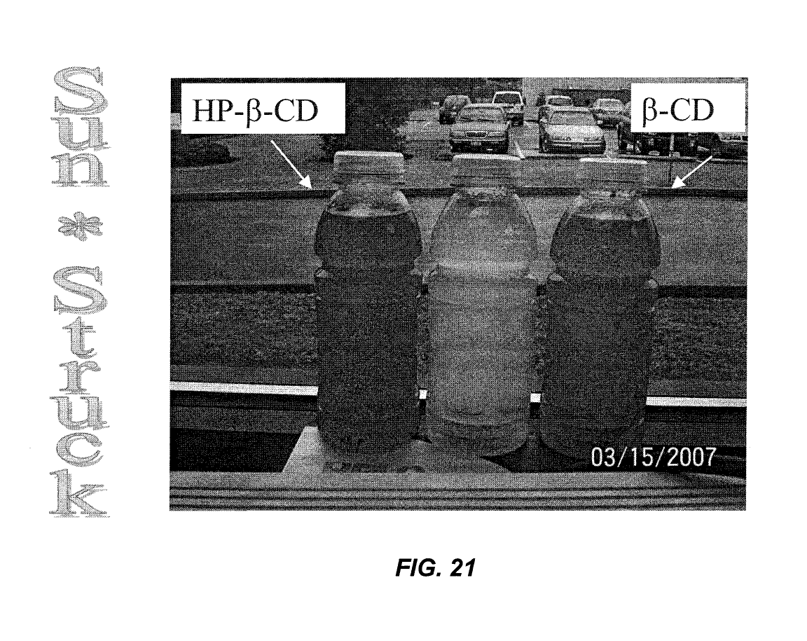

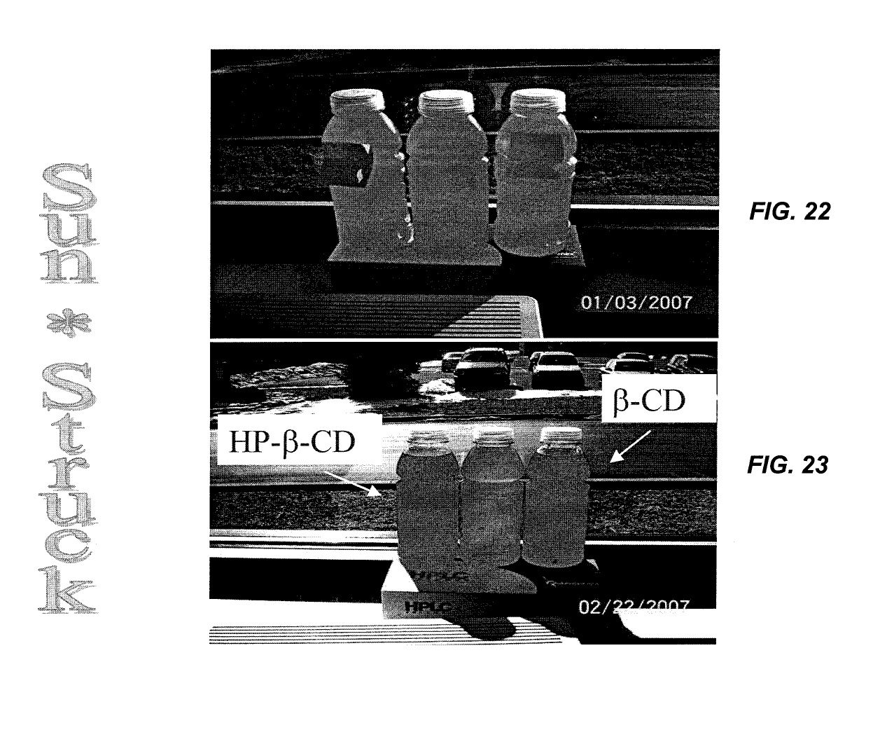

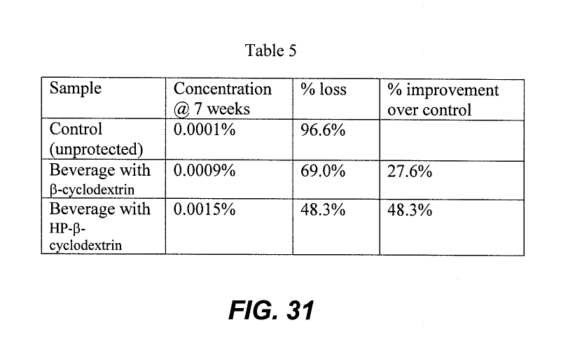

To a commercial vitamin fortified beverage (GLAC\check{E}AU multi-v lemonade (a-zinc)) purchased at a local grocery store is added 0.2 wt % of the “citral-cyclodextrin/HP β-cyclodextrin blend” from Example 19A and 0.01 wt % red 40 color. The mixture is returned to the original container and sealed. The sealed bottle is place in a south facing window for 5-6 weeks or until color changes are noted. Excess beverage is stored refrigerated in glass. Results are shown in Table 5 (FIG. 31). Visual images of the bottled beverages are shown in FIGS. 21-23. The bottles show that the red 40 color degrades upon exposure to light. The color stability is best with the hydroxyl propyl β-cyclodextrin.

### Example 40A

### Stabilization of Citral to Sunlight with Cyclodextrin

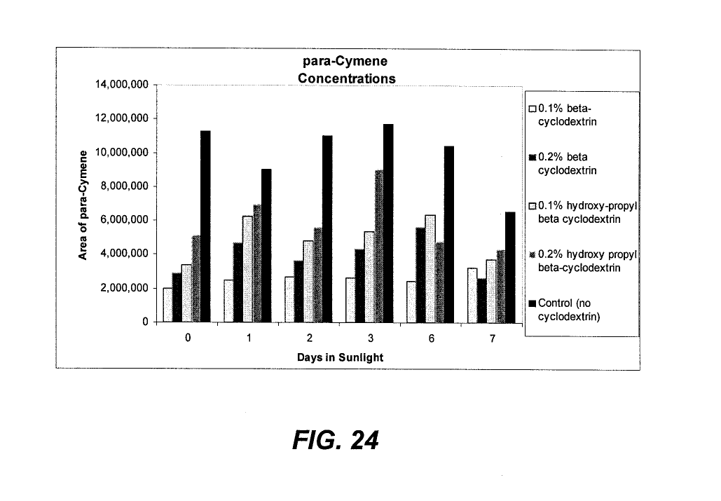

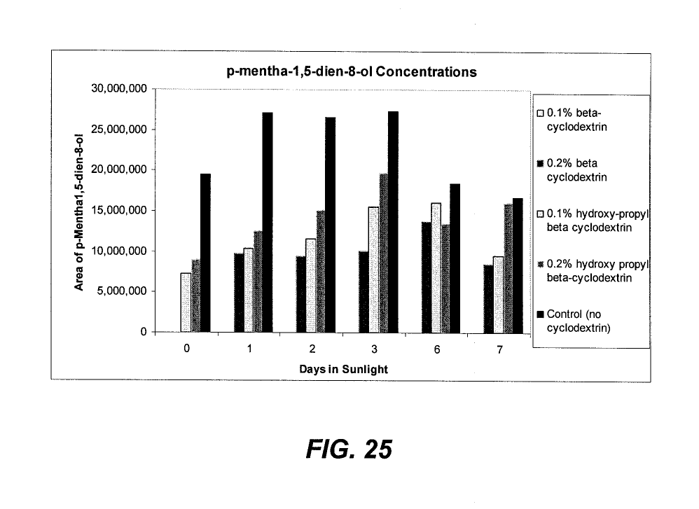

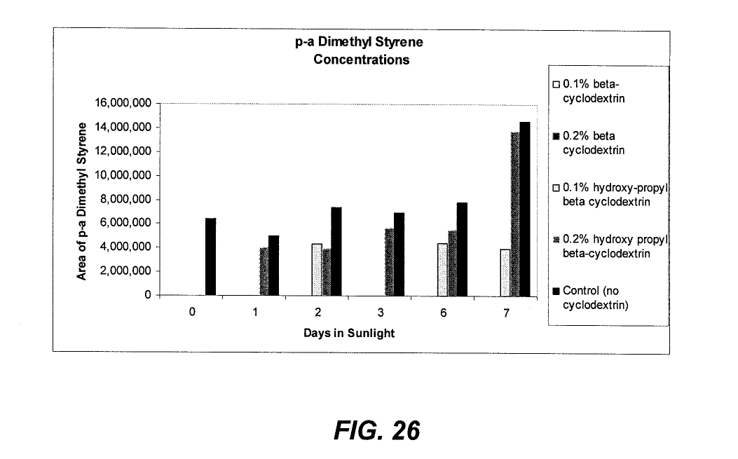

The encapsulated citral products of Example 19 were studied by exposure to summer sun for 7 days as in the above example, but with the goal of monitoring thermal and photo oxidation products using two different concentrations of cyclodextrins. We have previously demonstrated that photo-oxidation follows a similar reaction path as thermal oxidation in acidic media and is afforded protection using β-cyclodextrin; no such information exists for hydroxypropyl-β-cyclodextrin. The citral complexes were studied at two different concentrations and were designed to deliver a constant citral level when used at 0.1 wt % or 0.2 wt % in acidic beverage base consisting of 10% sucrose and 0.5% citric acid. A 0.1 wt % citral in ethanol was used as a control. 500 ml of each sample was prepared and divided into six (6) four (4) oz samples with no allowed headspace and sealed. Analysis was preformed using SPME and a LECO Pegasus III GC/TOF-MS as previously reported; samples were analyzed in triplicate using a “Latin Square” sampling protocol. Results for typical offnotes are displayed below in FIGS. 24-26. No p-α-dimethyl styrene was detected in either β-cyclodextrin protected beverage.

### Example 41

### Cyclodextrin Inclusion Complex with β-cyclodextrin and Furaneol, Pectin as an Emulsifier, and Process for Forming Same

At atmospheric pressure, in a 1-L reactor, 200 g of β-cyclodextrin was dry blended with 4.0 g of beet pectin (2 wt % of pectin: β-cyclodextrin; XPQ EMP 5 beet pectin available from Degussa-France) to form a dry blend. 500 g of deionized water was added to the dry blend of β-cyclodextrin and pectin to form a slurry or mixture. The 1-L reactor was set up for heating and cooling via a lab-scale water bath heating and cooling apparatus. The mixture was heated at 50 degrees C. for 0.5 hours and agitated by stirring. 150 g of 15% natural furaneol (4-hydroxy-2,5-dimethyl-3(2H) furanone) FEMA # 3174 in ethanol solution, available from Alfrebro, a division of Cargill, Monroe, Ohio was added. The reactor was sealed, and the resulting mixture was stirred for 4 hours at about 50 degrees C. The cooling portion of the heating and cooling lab apparatus was then turned on, and the mixture was stirred overnight at about 5-10 degrees C. The mixture was then spray dried on a BUCHI B-191 lab spray dryer (available from Buchi, Switzerland) having an inlet temperature of approximately 210 degrees C. and an outlet temperature of approximately 105 degrees C. A percent retention of 4.6 wt % of furaneol (45.5% yield) in the cyclodextrin inclusion complex was achieved.

### Example 42

### Flavor Composition Comprising Cyclodextrin-Encapsulated Furaneol and Excess Uncomplexed Cyclodextrin

Encapsulated furaneol was produced according to the method set forth in Example 41. The resulting dry powder including the cyclodextrin-encapsulated furaneol is dry blended with additional β-cyclodextrin to achieve a wt % of about 0.05 wt % of furaneol in the resulting dry powder mixture (“furaneol-cyclodextrin/cyclodextrin blend”). The furaneol-cyclodextrin/cyclodextrin blend is added to a Cream Soda beverage in a wt % of about 0.2 wt % of the dry powder mixture (i.e., β-cyclodextrin-encapsulated furaneol plus additional β-cyclodextrin) to the total weight of the beverage. This provides 5-10 ppm of furaneol and about 0.2 wt % of β-cyclodextrin to the acidic beverage. This composition is intended to protect flavor such as cis-3-hexenol, furaneol, vanillin, raspberry ketone ionones, etc; color, such as RED 40, and prevent the migration of flavor (and/or) other ingredients into the plastic container.

### Example 43

### Flavor Composition Comprising Cyclodextrin-Encapsulated Furaneol and Excess Uncomplexed Derivatized Cyclodextrin

Encapsulated furaneol was produced according to the method set forth in Example 41. The resulting dry powder including the cyclodextrin-encapsulated furaneol is dry blended with additional HP-β-cyclodextrin to achieve a wt % of about 0.05 wt % of furaneol in the resulting dry powder mixture (“furaneol-cyclodextrin/HP-β cyclodextrin blend”). The furaneol-cyclodextrin/HP-β cyclodextrin blend is added to a Cream Soda beverage in a wt % of about 0.2 wt % of the dry powder mixture (i.e., β-cyclodextrin-encapsulated furaneol plus additional β-cyclodextrin) to the total weight of the beverage. This provides 5-10 ppm of furaneol and about 0.2 wt of β-cyclodextrin to the acidic beverage. This composition is intended to protect flavor such as cis-3-hexenol, furaneol, vanillin, raspberry ketone ionones, etc; color, such as RED 40, and prevent the migration of flavor (and/or) other ingredients into the plastic container.

### Example 44

### Analytical Measurements

An initial analytical profile is generated for the formulations in Examples 39, 40, 42 and 43. A 2 ml sample is withdrawn from the container for headspace analysis as detailed in Example 37 (although different flavor compounds and offnotes will be analyzed). Vanillin and Red 40 concentrations will be monitored by HPLC with UV detection.

### Example 45

### Formation of Large Particle Cyclodextrin Inclusion Complexes with Arachidonic Acid (40%)

In an industrial mixer, (Kitchen Aid Proline, Kitchen Aid, St. Joseph, Mich.), equipped within a glove bag to provide an inert atmosphere, 1000.0000 g of □-cyclodextrin was mixed at low speed for 30 minutes with 800.0000 g of distilled water and 20.00 g beet pectin (2.0 wt % pectin, XPQ EMP 4 beet pectin available from Degussa-France) to form a paste in a dough mixture. The mixture was mixed at high speed for 2 minutes to remove any remaining dissolved air. 300.0000 g of arachidonic acid (40%) (Cargill, Minneapolis, Minn.) was added slowly while mixing for 90 minutes.

Mixture was transferred to two pans for vacuum drying. The entire process was carried out under a nitrogen atmosphere. The mixture dried at 79° C. and 0.1 torr for 9 hrs. The resulting product was a fine powder with 36 wt % retention ARA; no surface oils were noted.

### Example 46

### Use in Infant Formula

A cyclodextrin-encapsulated arachodonic acid produced according to Example 45 is incorporated into an infant formula.

All patents, publications and references cited herein are hereby fully incorporated by reference. In case of conflict between the present disclosure and incorporated patents, publications and references, the present disclosure should control.

1. A product comprising a guest complexed with a cyclodextrin and a guest degradation product, the product having a guest to guest degradation product ratio of at least about 5:1 when stored for at least about 30 days at a temperature of at least about 88° F., wherein the guest comprises a flavor.

2. The product of claim 1, wherein the guest to guest degradation product ratio is at least about 10:1 when stored for at least about 30 days at a temperature of at least about 88° F.

3.-13. (canceled)

14. The product according to claim 1, wherein the cyclodextrin comprises a β-cyclodextrin.

15. The product according to claim 14, wherein the β-cyclodextrin comprises a substituted β-cyclodextrin.

16. The product according to claim 15, wherein the substituted β-cyclodextrin comprises a hydroxypropyl β-cyclodextrin.

17. The product according to claim 16, wherein the β-cyclodextrin comprises a mixture of hydroxypropyl β-cyclodextrin and β-cyclodextrin.

18. The product according to claim 17, wherein the hydroxypropyl β-cyclodextrin and β-cyclodextrin are present in a ratio of about 2:1 to about 1:30.

19.-20. (canceled)

21. The product of claim 1, wherein the flavor comprises citral.

22.-26. (canceled)

27. The product according to claim 1, wherein the cyclodextrin is present in an amount of about 0.05 wt % to about 0.50 wt %.

28.-35. (canceled)

36. A method for improving the flavor stability of a product when exposed to light comprising adding a guest complexed with a cyclodextrin to the product, wherein the guest comprises a flavor and wherein the flavor stability is improved by at least about 25% as compared to a control.

37. The method according to claim 36, wherein the flavor stability is improved by at least about 45% as compared to a control.

38.-39. (canceled)

40. The method according to claim 36, wherein the guest is complexed with the cyclodextrin in the presence of an emulsifier.

41. The method according to claim 36, wherein the cyclodextrin comprises a β-cyclodextrin.

42. The method according to claim 41, wherein the β-cyclodextrin comprises a substituted β-cyclodextrin.

43. The method according to claim 42, wherein the substituted β-cyclodextrin comprises a hydroxypropyl β-cyclodextrin.

44. The method according to claim 41, wherein the β-cyclodextrin comprises a mixture of hydroxypropyl β-cyclodextrin and β-cyclodextrin.

45. The method according to claim 44, wherein the hydroxypropyl β-cyclodextrin and β-cyclodextrin are present in a ratio of about 2:1 to about 1:30.

46.-47. (canceled)

48. The method according to claim 36, wherein the flavor comprises citral.

49.-53. (canceled)

54. The method according to claim 36, wherein the cyclodextrin is present in an amount of about 0.05 wt % to about 0.50 wt %.

55.-58. (canceled)
